SEQUENCE LISTING (18 sequences)

SEQ ID NO: 1 - DNA, 24 bp, Artificial
  SOX2 forward primer
gggggaaagtagtttgctgcctct

SEQ ID NO: 2 - DNA, 20 bp, Artificial
  SOX2 reverse primer
cctcctctggccgatcctgc

SEQ ID NO: 3 - DNA, 24 bp, Artificial
  NANOG forward primer
cagcctccagcagatgcaagaact

SEQ ID NO: 4 - DNA, 21 bp, Artificial
  NANOG reverse primer
tgaggccttctgcgtcacacc

SEQ ID NO: 5 - DNA, 21 bp, Artificial
  Oct4 forward primer
agcaaaacccggaggagtccc

SEQ ID NO: 6 - DNA, 25 bp, Artificial
  Oct4 reverse primer
gcagatggtcgtttggctgaatacc

SEQ ID NO: 7 - DNA, 24 bp, Artificial
  MIXL1 forward primer
aaactgagaagtatcctctgctaa

SEQ ID NO: 8 - DNA, 24 bp, Artificial
  MIXL1 reverse primer
tcttctgcaagcctccctaacaca

SEQ ID NO: 9 - DNA, 22 bp, Artificial
  BRACHY forward primer
atgagcctcgaatccacatagt

SEQ ID NO: 10 - DNA, 22 bp, Artificial
  BRACHY reverse primer
tcctcgttctgataagcagtca

SEQ ID NO: 11 - DNA, 20 bp, Artificial
  C/EBP-alpha forward primer
gagggaccggagttatgaca

SEQ ID NO: 12 - DNA, 20 bp, Artificial
  C/EBP-alpha reverse primer
ttcacattgcacaaggcact

SEQ ID NO: 13 - DNA, 19 bp, Artificial
  PU.1 forward primer
gacaggcagcaagaagaag

SEQ ID NO: 14 - DNA, 19 bp, Artificial
  PU.1 reverse primer
ttggacgagaactggaagg

SEQ ID NO: 15 - DNA, 21 bp, Artificial
  SCL forward primer
caaagttgtgcggcgtatctt

SEQ ID NO: 16 - DNA, 23 bp, Artificial
  SCL reverse primer
tcattcttgctgagcttcttgtc

SEQ ID NO: 17 - DNA, 21 bp, Artificial
  GAPDH forward primer
ggagcgagatccctccaaaat

SEQ ID NO: 18 - DNA, 23 bp, Artificial
  GAPDH reverse primer
ggctgttgtcatacttctcatgg

(19) United States
(12) Patent Application Publication
(10) Pub. No.: US 2022/0186186 A1
(43) Pub. Date: Jun. 16, 2022

(54) COMPOSITION FOR INDUCING DIRECT CONVERSION OF SOMATIC CELL INTO COMMON MYELOID PROGENITOR AND USE THEREOF

(71) Applicant: KOREA INSTITUTE OF SCIENCE AND TECHNOLOGY, Seoul (KR)

(72) Inventors: So Yeon KIM, Seoul (KR); Eun Gyeong YANG, Seoul (KR); HyeRan GWAK, Seoul (KR)

(73) Assignee: KOREA INSTITUTE OF SCIENCE AND TECHNOLOGY, Seoul (KR)

(21) Appl. No.: 17/387,782

(22) Filed: Jul. 28, 2021

(30) Foreign Application Priority Data
Dec. 15, 2020 (KR) 10-2020-0175828

(51) Int. Cl. C12N 5/0786; A61K 35/15

(52) U.S. Cl. CPC C12N 5/0645; A61K 35/15; C12N 2501/2304; C12N 2501/22; C12N 2501/999

(57) ABSTRACT

Provided are: a composition for inducing direct conversion from somatic cells into common myeloid progenitor cells, the composition including a chemical cocktail; a method of direct conversion of somatic cells into common myeloid progenitor cells and macrophages by using the composition; common myeloid progenitor cells or macrophages prepared by the method; a pharmaceutical composition for preventing or treating fibrosis or scars, cell therapeutics, a composition for screening drugs, and a 3D printable biomaterial composition for fabricating artificial tissues, each using the common myeloid progenitor cells or the macrophages.

### CROSS-REFERENCE TO RELATED APPLICATION

This application claims the benefit of Korean Patent Application No. 10-2020-0175828, filed on Dec. 15, 2020, in the Korean Intellectual Property Office, under 35 U.S.C. § 119, the entire disclosures of which is incorporated herein in its entirety by reference.

### BACKGROUND

### 1. Field

One or more embodiments relate to a composition for inducing direct conversion of somatic cells into common myeloid progenitor cells, to a method of direct conversion of somatic cells into common myeloid progenitor cells and macrophages by using the composition, and to a use of the common myeloid progenitor cells and the macrophages for the prevention or treatment of fibrosis or scars.

### 2. Description of Related Art

Existing methods for macrophage differentiation using embryonic stem cells or induced pluripotent stem cells require establishing embryonic stem cells by destroying embryos, or require somatic cells to go through a de-differentiation process to revert back to an induced pluripotent stem cell stage, before being differentiated into macrophages. Therefore, the use of embryonic stem cells may give rise to ethical issues. Further, the use of induced pluripotent stem cells, despite the time, monetary costs, and efforts it consumes, provides low yields in the step in which differentiation is carried out, and suffers from the disadvantage of being inefficient due to the difficulty in artificially regulating potency. Further, the use of induced pluripotent stem cells is highly likely to give rise to the formation of teratomas derived from undifferentiated cells, thus giving rise to safety issues surrounding their use.

In this respect, one previous study has confirmed that Oct4 transduction induced direct conversion of human fibroblasts into blood progenitors resulting in ability to differentiate toward monocytes, neutrophils, dendritic cells, or macrophages (Szabo, E., et al. (2010). “Direct conversion of human fibroblasts to multilineage blood progenitors.” Nature, 468(7323), 521-526. doi:10.1038/nature09591). However, the methods using gene transduction, such as the above, present safety issues concerning genetic manipulation.

Therefore, it is necessary to develop a technique capable of inducing direct conversion from somatic cells into macrophages without genetic manipulation.

### SUMMARY

One or more embodiments include a composition, including a chemical or a chemical cocktail, for inducing direct conversion from somatic cells into common myeloid progenitor (CMP) cells.

One or more embodiments include a method of direct conversion of somatic cells into CMP cells and macrophages.

One or more embodiments include CMP cells or macrophages, prepared by the method of direct conversion.

One or more embodiments include a composition including CMP cells or macrophages prepared by the method of direct conversion.

One or more embodiments include a pharmaceutical composition for preventing or treating fibrosis or scars, including the composition for inducing direct conversion or one or more selected from CMP cells and macrophages prepared by the method of direct conversion.

One or more embodiments include a method of preventing or treating fibrosis or scars, including administering an effective amount of the pharmaceutical composition to a subject.

One or more embodiments include cell therapeutics for preventing or treating fibrosis or scars, including one or more selected from CMP cells and macrophages prepared by the method of direct conversion.

One or more embodiments include a composition for screening drugs for preventing or treating fibrosis or scars, the composition including one or more selected from CMP cells and macrophages prepared by the method of direct conversion.

One or more embodiments include a 3D printable biomaterial composition for fabricating artificial tissues for the treatment of fibrosis or scars, the composition including either the above composition for inducing direct conversion from somatic cells into CMP cells, or one or more selected from CMP cells and macrophages prepared by the method of direct conversion.

Additional aspects will be set forth in part in the description which follows and, in part, will be apparent from the description, or may be learned by practice of the presented embodiments.

One aspect provides a composition for inducing direct conversion from somatic cells into common myeloid progenitor (CMP) cells, the composition including a chemical or a chemical cocktail.

The term “direct conversion (direct reprogramming, transdifferentiation)” refers to a process of inducing conversion between mature (terminally differentiated) cells of completely different cell types in a higher organism (Kim, J. et al, Neurobiol. 22, 778-784, 2012). Unlike conventional techniques that require reprogramming to induced pluripotent stem cells (iPSCs) and re-differentiating the same to desired cells, direct conversion differs therefrom in that it induces conversion that is directly toward desired cells without going through an iPSC stage. Currently, direct conversion is acknowledged for its potential applications in disease modeling, drug development, etc. and is also known to be a technique applicable to gene therapy, regenerative medicine, and the like.

The term “chemical compound” used herein may be one chemical compound or a chemical cocktail.

The chemical compound may enhance the expression of SRY(sex determining region Y)-box 2 (SOX2). Compared to gene-based direct conversion methods using the transduction of SOX2, chemical-based direct conversion methods are not only safer, but also can reduce the direct conversion time.

The term “chemical cocktail” used herein can be interchangeably used with “chemical composition” and refers to a combination of two or more compounds. However, the chemical cocktail does not exclude the use of a single compound. In some specific embodiments, the chemical cocktail may be a cocktail of small molecule compounds. In some specific examples, the chemical cocktail may include one or more compounds.

The composition of chemical cocktail may be commercially available drugs, and accordingly, excellent stability and safety may be ensured.

The chemical compound includes a TGF-β receptor inhibitor. The composition may include the TGF-β receptor inhibitor and can thus induce direct conversion from somatic cells to CMP cells.

The chemical cocktail may further include a histone deacetylase (HDAC) inhibitor, a glycogen synthase kinase 3 (GSK-3) inhibitor, or a combination thereof. The composition including a TGF-β receptor inhibitor may further include an HDAC inhibitor, a GSK-3 inhibitor, or a combination thereof, to thereby have enhanced capability for direct conversion from somatic cells to CMP cells.

The chemical cocktail may include two or more selected from among a transforming growth factor β (TGF-β) receptor inhibitor, a histone deacetylase (HDAC) inhibitor, and a glycogen synthase kinase 3 (GSK-3) inhibitor. In some specific embodiments, the chemical cocktail may include a TGF-β receptor inhibitor and an HDAC inhibitor. In some specific embodiments, the chemical cocktail may include all of a TGF-β receptor inhibitor, an HDAC inhibitor, and a GSK-3 inhibitor.

The TGF-β receptor may be a TGF-β receptor type I (TGF-β RI, TGFβRI, TGFBR1, and ALK5).

The TGF-β receptor inhibitor may be a TGF-β receptor type I inhibitor (TGF-β RI kinase inhibitor, ALK5 inhibitor). The TGF-β receptor inhibitor may be a TGF-β receptor type I inhibitor II (TGF-β RI kinase inhibitor II, ALK5 inhibitor II).

In some specific embodiments, the TGF-β receptor inhibitor may be, but is not limited to, 2-[3-(6-methylpyridin-2-yl)-1H-pyrazol-4-yl]-1,5-naphthyridine (616452), SB431542, galunisertib (LY2157299), LY3200882, vactosertib (TEW-7197), PF-06952229, or a combination of two or more thereof. In some specific embodiments, the TGF-β receptor inhibitor may be 616452.

The HDAC inhibitor may be, but is not limited to, a valproate, Trichostatin A, phenylbutyrate, sodium butyrate, suberoylanilide hydroxamic acid (SAHA), suberohydroxamic acid (SBHA), or a combination of two or more thereof. In some specific embodiments, the HDAC inhibitor may be a valproate.

The valproate may be valproic acid (VPA), sodium valproate, divalproex sodium, or a combination of two or more thereof. In some specific embodiments, the valproate may be VPA.

The “valproic acid (VPA)” is also known as “2-propylpentanoic acid” and is listed on the WHO Essential Medicines List.

The “glycogen synthase kinase 3 (GSK-3)” is one of phosphorylases conserved in eukaryotes and transfers phosphate groups onto serine and threonine amino acid residues. The GSK-3 exists in two forms, GSK-3a and GSK-3β.

The “GSK-3 inhibitor” may refer to a substance that inhibits the activity of GSK-3.

In some specific embodiments, the GSK-3 inhibitor may be a GSK-3β inhibitor.

In some specific embodiments, the GSK-3 inhibitor may be, but is not limited to, 6-((2-((4-(2,4-dichlorophenyl)-5-(4-methyl-1H-imidazol-2-yl)pyrimidin-2-yl)amino)ethyl)amino)nicotinonitrile (CHIR99021), TD114-2, SB216763, SB415286, or a combination of two or more thereof.

The chemical cocktail may optionally further include an antioxidant. Accordingly, in some specific embodiments, the chemical cocktail may include a TGF-β receptor inhibitor, an HDAC inhibitor, and an antioxidant. In some specific embodiments, the chemical cocktail may include a TGF-β receptor inhibitor, an HDAC inhibitor, a GSK-3 inhibitor, and an antioxidant. When the chemical cocktail further includes an antioxidant, conversion efficiency may be increased.

The “antioxidant” refers to a substance that helps a human body protect itself from oxidative stress by removing reactive oxygen species. The antioxidant may include naturally occurring substances as well as artificially synthesized substances. The antioxidant includes polyphenols including catechin, vitamins (ex. vitamin A, vitamin E, and vitamin C), selenium, coenzyme Q10, and the like, but is not limited to the aforementioned types.

In some specific embodiments, the antioxidant may be, but is not limited to, ascorbic acid, resveratrol, N-acetylcysteine, ethylbisiminomethylguaiacol manganese chloride (EUK-134), an NADPH oxidase inhibitor, or a combination of two or more thereof. In some specific embodiments, the antioxidant may be ascorbic acid.

The “ascorbic acid” is also known as “vitamin C” and is one of water-soluble vitamins.

The “resveratrol” is a type of polyphenol, and can be discovered in many plants including berries and the like.

The term “somatic cells” refers to all cells excluding reproductive cells. The somatic cells, for example, may be derived or isolated from a mammal such as a human, a horse, a sheep, a pig, a goat, a camel, an antelope, a dog, and the like.

The expression “isolation of cells” may refer to removal of at least 20%, 30%, 40%, 50%, 60%, 70%, 80%, 90%, 95%, or 99% of cells that are normally connected to the cells sought to be isolated in untreated tissues. A cell group including cells obtained from a tissue may be referred to as being “isolated” when other cells that are normally connected to these cells constitute less than 50% of the total number of cells inside the tissue in an untreated state. In the present specification, the expression “isolated” may be used to refer to tissues or cells that exist in a different environment than the environment of naturally emerging tissues or cells. For example, cells naturally emerge in a multicellular organ, and if these cells are removed from the multicellular organ, the cells are considered to be “isolated”.

In some specific embodiments, the somatic cells may be, but are not limited to, one or more selected from fibroblasts, adipose stromal cells, epithelial cells, muscle cells, oral epithelial cells, somatic cells extracted from urine, blood cells, hair follicle stem cells, neural stem cells, hematopoietic stem cells, and mesenchymal stem cells.

In some specific embodiments, the somatic cells may be fibroblasts. The term “fibroblast” used herein refers to a type of cell constituting a component of fibrous connective tissue and may refer to a cell of connective tissue in a mammal.

The term “common myeloid progenitor (CMP) cell” is also known as “common myeloid stem cell” and refers to a type of cell that is able to differentiate to various myeloid cells. Hematopoietic stem cells (HSCs) are stem cells capable of generating blood cells of all types. The process through which blood cells of all types are formed from HSCs is referred to as hematopoiesis. The HSCs can differentiate to CMP cells or common lymphoid progenitor (CLP) cells. In some specific embodiments, the CMP cells are induced by direct conversion from somatic cells. Accordingly, by using the composition according to one aspect, direct conversion from somatic cells into CMP cells may be induced without passing through HSCs.

The CMP cells are able to differentiate into various myeloid cells, and for example, may differentiate into myeloblasts, basophils, neutrophils, eosinophils, monocytes, granulocytes, dendritic cells, or macrophages, but are not limited thereto. In some specific embodiments, the CMP cells may differentiate to macrophages. Accordingly, the composition may be used as a composition for inducing direct conversion from somatic cells to macrophages.

The term “macrophage” is a type of white blood cell and has phagocytic function. Macrophages use phagocytosis to break down cell debris, foreign material, bacteria, cancer cells, abnormal proteins, and the like. Also, macrophages may play an important role in tissue regeneration, cell regeneration, or wound healing, by secreting cytokines, removing damaged tissue, or the like. Therefore, conditions caused by dysfunctional fibroblast regeneration, such as fibrosis, keloids, and hypertrophic scars, may be treated by inducing direct conversion from fibroblasts to macrophages.

The composition may include the chemical cocktail in an amount effective for inducing direct conversion from somatic cells to CMP cells.

In detail, the method may include preparing CMP cells by culturing somatic cells in media containing a TGF-β receptor inhibitor. The media may further include an HDAC inhibitor, a GSK-3 inhibitor, an antioxidant, or a combination thereof. When the media further contain an HDAC inhibitor, a GSK-3 inhibitor, or a combination thereof, the efficiency of direct conversion from somatic cells to CMP cells may be increased. When the media further contain an antioxidant, conversion efficiency may be enhanced.

In detail, the method may include preparing CMP cells by culturing somatic cells in media containing a TGF-β receptor inhibitor and an HDAC inhibitor.

The composition for inducing direct conversion from somatic cells to CMP cells may be a kit composition or a cell culture medium composition.

The kit may be prepared as a plurality of separate packaging or compartments that contain the above-described chemical components.

The kit may include instructions providing an appropriate concentration range and administration timing according to the type of cells induced.

Another aspect provides a method of direct conversion of somatic cells into CMP cells and macrophages.

The method includes preparing CMP cells by culturing somatic cells in media containing the composition according to the one aspect.

More specifically, the method may include preparing CMP cells by culturing somatic cells in media containing a TGF-β receptor inhibitor, an HDAC inhibitor, and a GSK-3 inhibitor.

The method may be carried out in vitro or in vivo.

The above method of direct conversion using a chemical cocktail may produce CMP cells and macrophages with higher yields in a shorter period of time, compared with direct conversion methods using SOX2 transduction. Also, by using drugs that are actually clinically applied, the above method allows a safer direct conversion without genetic manipulation.

In the preparation of CMP cells, the media may further include an antioxidant.

The preparation of CMP cells may be subdivided into two steps. Accordingly, the method may include: conducting first culturing of somatic cells in media containing a TGF-β receptor inhibitor; and conducting second culturing of the cultured somatic cells in media containing a TGF-β receptor inhibitor, and a GSK-3 inhibitor.

One or more from the media of the first culturing and the media of the second culturing may further include an HDAC inhibitor, an antioxidant, or a combination thereof. In some specific embodiments, the media of the first culturing may include a TGF-β receptor inhibitor, an HDAC inhibitor, and an antioxidant. In some specific embodiments, the media of the second culturing may include a TGF-β receptor inhibitor, an HDAC inhibitor, a GSK-3 inhibitor, and an antioxidant.

In the preparation of CMP cells, the concentration of the TGF-β receptor inhibitor may be selected to an appropriate concentration that is sufficient to induce direct conversion, depending on the specific type of the TGF-β receptor inhibitor. In the media, the concentration of the TGF-β receptor inhibitor may be selected in a range of about 0.01 μM to about 1,000 μM, but is not limited thereto. In some specific embodiments where the TGF-β receptor inhibitor is 616452, the concentration of 616452 in the media may be from about 1 μM to about 20 μM, about 3 μM to about 20 μM, about 3 μM to about 18 μM, about 3 μM to about 15 μM, about 3 μM to about 12 μM, about 5 μM to about 20 μM, about 5 μM to about 18 μM, about 5 μM to about 15 μM, about 5 μM to 12 μM, or about 10 μM. If the concentration of the TGF-β receptor inhibitor is too high, cells may die. If the concentration of the TGF-β receptor inhibitor is too low, conversion efficiency may be decreased. For example, if the concentration of 616452 is less than 3 μM, the direct conversion may not occur properly.

In the preparation of CMP cells, the concentration of the HDAC inhibitor in the media may be selected to be an appropriate concentration that is sufficient to induce direct conversion, depending on the specific type of the HDAC inhibitor. In the media, the concentration of the HDAC inhibitor may be selected in a range of about 0.01 mM to about 100.0 mM, but is not limited thereto. In some specific embodiments, if the HDAC inhibitor is VPA, the concentration of VPA in the media may be about 0.01 mM to 10.0 mM, about 0.01 mM to about 5.0 mM, about 0.01 mM to about 1.0 mM, about 0.1 mM to about 10.0 mM, about 0.1 mM to about 5.0 mM, about 0.1 mM to about 1.0 mM, or about 0.5 mM. When the concentration of the HDAC inhibitor departs from the aforementioned ranges, the direct conversion may not occur properly, or conversion efficiency may be decreased.

In the preparation of CMP cells, the concentration of the GSK-3 inhibitor in the media may be selected to be an appropriate concentration that is sufficient to induce direct conversion, depending on the specific type of the GSK-3 inhibitor. In the media, the concentration of the GSK-3 inhibitor may be selected in a range of about 0.01 μM to about 1,000 μM, but is not limited thereto. In some specific embodiments, when the GSK-3 inhibitor is CHIR99021, the concentration of CHIR99021 in the media may be about 0.1 μM to about 10 μM, about 0.1 μM to about 5 μM, about 0.5 μM to about 10 μM, about 0.5 μM to about 5 μM, about 1 μM to about 10 μM, about 1 μM to about 5 μM, or about 3 μM. If the concentration of the GSK-3 inhibitor is too high, cells may die. If the concentration of the GSK-3 inhibitor is too low, conversion efficiency may be decreased.

In the preparation of CMP cells, the concentration of the antioxidant in the media may be selected to be an appropriate concentration that is sufficient to increase the conversion efficiency of direct conversion, depending on the specific type of the antioxidant. In the media, the concentration of the antioxidant may be selected within a range of about 0.001 μg/ml to about 1,000 μg/ml, but is not limited thereto. In some specific embodiments where the antioxidant is ascorbic acid, the concentration of ascorbic acid in the media may be about 10 μg/ml to about 1000 μg/ml, about 10 μg/ml to about 500 μg/ml, about 10 μg/m to about 300 μg/ml, about 10 μg/ml to about 200 μg/ml, about 50 μg/ml to about 1000 μg/ml, about 50 μg/ml to about 500 μg/ml, about 50 μg/ml to about 300 μg/ml, about 50 μg/ml to about 200 μg/ml, about 50 μg/ml, or about 100 μg/ml. In some specific embodiments where the antioxidant is resveratrol, the concentration of the resveratrol in the media may be about 0.01 μM to about 50 μM, or about 0.01 μM to about 20 μM, but is not limited thereto. Too high or too low a concentration of the antioxidant may decrease conversion efficiency and may induce apoptosis.

In the preparation of CMP cells, the media include all media that are commonly used for somatic cell culture in the relevant technical field. Media used for culturing may generally include a carbon source, a nitrogen source, and trace element components. Also, the media may include a component conducive to inducing direct conversion from somatic cells to CMP cells. In some specific embodiments, the media may be reprogramming media. In some specific embodiments, the media may include Knockout Serum Replacement (KSR), penicillin/streptomycin, glutamine, non-essential amino acids, IGFII, bFGF2, β-mercaptoethanol, or a combination of two or more thereof, but are not limited thereto.

In the preparation of CMP cells, the media may be one or more selected from DMEM (Dulbecco's Modified Eagle's Medium), MEM (Minimal Essential Medium), BME (Basal Medium Eagle), RPMI 1640, DMEM/F-10 (Dulbecco's Modified Eagle's Medium: Nutrient Mixture F-10), DMEM/F-12 (Dulbecco's Modified Eagle's Medium: Nutrient Mixture F-12), α-MEM (α-Minimal essential Medium), G-MEM (Glasgow's Minimal Essential Medium), IMDM (Isocove's Modified Dulbecco's Medium), and KnockOut DMEM.

The somatic cells may be one or more selected from fibroblasts, adipose stromal cells, epithelial cells, muscle cells, oral epithelial cells, somatic cells extracted from urine, blood cells, hair follicle stem cells, neural stem cells, hematopoietic stem cells, and mesenchymal stem cells, but are not limited thereto.

Culture conditions capable of inducing direct conversion of the somatic cells may be selected from culture conditions commonly adapted for inducing direct conversion of somatic cells in the relevant field.

The culturing may be subculturing. The first culturing and the second culturing may be subculturing. The term “subculturing” refers to a process through which a portion of cells in a source culture is transferred to a new culture medium to culture new cells.

The culturing may be adherent-culturing. The first culturing and the second culturing may be adherent-culturing. For adherent-culturing, cells may be attached and cultured on a geltrex- or matrigel-coated cell support, for example, plate.

The culturing time may be a duration sufficient for directly converting somatic cells to CMP cells, and may be, for example, about 20 days to about 36 days, about 20 days to about 32 days, about 24 days to about 36 days, about 24 days to about 32 days, or about 28 days.

The first culturing time may be about 10 days to about 20 days, about 10 days to about 18 days, about 10 days to about 16 days, about 12 days to about 18 days, about 12 days to about 16 days, or about 14 days.

The second culturing time may be about 10 days to about 20 days, about 10 days to about 18 days, about 10 days to about 16 days, about 12 days to about 18 days, about 12 days to about 16 days, or about 14 days.

Cells prepared in the preparing of CMP cells may express one or more markers between CD45 and CD14. In an embodiment, cells prepared in the preparing of CMP cells did not express CD34 and thus could be confirmed that they are not hematopoietic stem cells; and alternatively, the cells demonstrated the expression of CD45 and CD14 and thus could be confirmed that they are CMP cells. Also, the above method exhibited superior conversion efficiency compared with that of a direct conversion method using SOX2 transduction.

The above method may further include differentiating the prepared CMP cells into myeloid cells.

The myeloid cells may be myeloblasts, basophils, neutrophils, eosinophils, monocytes, granulocytes, dendritic cells, or macrophages, but are not limited thereto.

In the differentiation into myeloid cells, the CMP cells may be cultured in media for differentiating the CMP cells to desired cells. Related art document (Szabo, E., et al. (2010). Nature, 468(7323), 521-526.; and J. Pulecio et al., Stem Cells. 2014 Nov. 32(11):2923-2938) suggests examples where CMP cells demonstrating CD45 expression are cultured in media for differentiating the same to desired cells, and thus differentiate to neutrophils, monocytes, granulocytes, dendritic cells, macrophages, and the like. For example, when the desired cells are macrophages, the media may be macrophage differentiation media.

In some specific embodiments, the method may further include differentiating the prepared CMP cells into macrophages.

In the differentiation into macrophages, the CMP cells may be cultured in macrophage differentiation media.

In some specific embodiments, in the differentiation into macrophages, the CMP cells may be cultured in media containing a macrophage colony stimulating factor (M-CSF), IL-4, or a combination thereof.

In the differentiation into macrophages, the media may be one or more selected from Dulbecco's Modified Eagle's Medium (DMEM), Minimal Essential Medium (MEM), Basal Medium Eagle (BME), RPMI 1640, DMEM/F-10 (Dulbecco's Modified Eagle's Medium: Nutrient Mixture F-10), DMEM/F-12 (Dulbecco's Modified Eagle's Medium: Nutrient Mixture F-12), α-Minimal essential Medium (α-MEM), Glasgow's Minimal Essential Medium (G-MEM), Isocove's Modified Dulbecco's Medium (IMDM), and KnockOut DMEM.

In the differentiation into macrophages, the culturing time may be about 2 days to about 14 days, about 2 days to about 10 days, about 2 days to about 8 days, about 4 days to about 14 days, about 4 days to about 10 days, about 4 days to about 8 days, about 6 days to about 14 days, about 6 days to about 10 days, about 6 days to about 8 days, or about 7 days.

The macrophages prepared in the differentiation into macrophages may be macrophages with phagocytic function. In an embodiment, it could be confirmed that the macrophages prepared in the differentiation into macrophages had phagocytic function.

The method may further include pretreating the somatic cells prior to the preparation of CMP cells.

The pretreatment may include: (1) culturing the somatic cells in media containing an antioxidant; and (2) adding an HDAC inhibitor to the media and culturing the somatic cells therein.

In the pretreatment, the media may include FBS, penicillin streptomycin (P/S), glutamine, non-essential amino acids, β-mercaptoethanol, or a combination of two or more thereof, but are not limited thereto.

In the pretreatment, the media may be one or more selected from Dulbecco's Modified Eagle's Medium (DMEM), Minimal Essential Medium (MEM), Basal Medium Eagle (BME), RPMI 1640, DMEM/F-10 (Dulbecco's Modified Eagle's Medium: Nutrient Mixture F-10), DMEM/F-12 (Dulbecco's Modified Eagle's Medium: Nutrient Mixture F-12), α-Minimal essential Medium (α-MEM), Glasgow's Minimal Essential Medium (G-MEM), Isocove's Modified Dulbecco's Medium (IMDM), and KnockOut DMEM.

In process (1), the culturing time may be a duration sufficient for cell pretreatment and may be, for example, about 12 hours to about 36 hours, about 12 hours to about 32 hours, about 12 hours to about 28 hours, about 16 hours to about 36 hours, about 16 hours to about 32 hours, about 16 hours to about 28 hours, about 20 hours to about 36 hours, about 20 hours to about 32 hours, about 20 hours to about 28 hours, or about 24 hours.

In process (2), the culturing time may be a duration sufficient for cell pretreatment and may be, for example, about 12 hours to about 36 hours, about 12 hours to about 32 hours, about 12 hours to about 28 hours, about 16 hours to about 36 hours, about 16 hours to about 32 hours, about 16 hours to about 28 hours, about 20 hours to about 36 hours, about 20 hours to about 32 hours, about 20 hours to about 28 hours, or about 24 hours.

Including the pretreatment may increase the efficiency of direct conversion of somatic cells.

Another aspect provides CMP cells, myeloid cells, or macrophages prepared by the above method of direct conversion.

Details about the CMP cells, the myeloid cells, and the macrophages are as described above.

The CMP cells and the macrophages may be utilized for various purposes, such as for preventing or treating fibrosis or scars.

Since these CMP cells and macrophages are induced by direct conversion from somatic cells without going through a pluripotent state of induced pluripotent stem cells, they are less likely to form teratomas derived from undifferentiated cells, and thus may be safe.

Another aspect provides a composition including the CMP cells, myeloid cells, or macrophages prepared by the above method of direct conversion.

In some specific embodiments, the composition provides a composition including the CMP cells or macrophages prepared by the above method of direct conversion.

Details about the CMP cells, the myeloid cells, and the macrophages are as described above.

The composition may be a preparation formulated with a carrier for administration to a subject by topical application to the skin, oral administration, injection, in vivo transplantation, or a tissue-engineered matrix. The carrier may be a pharmaceutically acceptable carrier. The pharmaceutically acceptable carrier may be, for example, saline solution, sterile water, Ringer's solution, buffered saline solution, dextrose solution, maltodextrin solution, glycerol, ethanol, human serum albumin (HSA), and a mixture of one or more thereof; and other common additives, such as an antioxidant, a buffer solution, and a bacteriostatic agent, may be also added as needed.

Another aspect provides a pharmaceutical composition for preventing or treating fibrosis or scars, including the composition for inducing direct conversion or one or more selected from CMP cells and macrophages prepared by the above method of direct conversion.

The term “fibrosis” refers to formation of excessive fibrous connective tissues in an organ or tissue during regeneration or reaction processes. Examples of such fibrosis include pulmonary edema, hepatocirrhosis, endomyocardial fibrosis, mediastinal fibrosis, myelofibrosis, retroperitoneal fibrosis, progressive massive fibrosis, nephrogenic systemic fibrosis, Crohn's disease, keloids, myocardial infarction, systemic sclerosis, and the like.

The term “scar” refers to a mark left on the skin that is healed from a damage by a disease or an injury. The scar includes, but is not limited to, an atrophic scar, a keloid, a hypertrophic scar, and the like. The cause of the scar is not particularly limited.

The term “prevention” includes suppressing the emergence of a disease.

The term “treatment” includes suppressing, reducing, or eliminating the progression of a disease.

The pharmaceutical composition may include, as an active ingredient, the composition for inducing direct conversion or one or more selected from CMP cells and macrophages, prepared by the above method of direct conversion.

The term “including/comprising as an active ingredient” refers to including an active component at a level that is capable of expressing the aforementioned effect. Also, this may include being formulated in various forms by addition of various components as supplementary components, for drug delivery, stabilization, or the like.

The pharmaceutical composition may further include one or more from known active ingredients having an effect of preventing or treating fibrosis or scars.

The pharmaceutical composition may further include a pharmacologically acceptable diluent or carrier. The diluent may be lactose, corn starch, soybean oil, microcrystalline cellulose, or mannitol, and may include, as a lubricant, magnesium stearate, talc, or a combination thereof. The carrier may be an excipient, a disintegrating agent, a binder, a lubricant, or a combination thereof. The excipient may be microcrystalline cellulose, lactose, low-substituted hydroxycellulose, or a combination thereof. The disintegrating agent may be calcium carboxymethylcellulose, sodium starch glycolate, anhydrous dibasic calcium phosphate, or a combination thereof. The binder may be polyvinylpyrrolidone, low-substituted hydroxypropyl cellulose, hydroxypropyl cellulose, or a combination thereof. The lubricant may be magnesium stearate, silicon dioxide, talc, or a combination thereof.

The composition may be administered in various oral or non-oral formulations for actual clinical administration; and when formulated, may be prepared with commonly used excipients or diluents, such as a filler, an extender, a binder, a humectant, a disintegrating agent, a surfactant, etc. wherein suitable formulations known in the relevant technical field may be preferably selected from ones disclosed in literature (Remington's Pharmaceutical Science, latest edition, Mack Publishing Company, Easton Pa.).

Another aspect provides a method of preventing or treating fibrosis or scars, including administering an effective amount of the pharmaceutical composition to a subject.

A preferable dose of the composition, although it varies depending on the subject's state and body weight, severity of the disease, a drug form, and administration route and duration, may be appropriately selected by an ordinary person in the art. For preferable effects, the macrophages induced by direct conversion according to a specific embodiment, with respect to an adult patient having a body weight of 70 kg, may be administered about 1,000-10,000 cells/dose, 1,000-100,000 cells/dose, 1,000-1000,000 cells/dose, 1,000-10,000,000 cells/dose, 1,000-100,000,000 cells/dose, 1,000-1,000,000,000 cells/dose, or 1,000-10,000,000,000 cells/dose, and may be administered as multiple doses at regular time intervals from once to several times a day, or may be administered multiple times at regular time intervals.

The composition may be administered to a subject by various routes. All modes of administration may be predictable. For example, the administration may be made by oral, rectal or intravenous, intramuscular or subcutaneous injection, and may be made by any route that involves topical application to the skin.

The term “subject” refers to any subject in need of treatment for fibrosis or scars, and more specifically, includes a mammal such as a human or non-human primate, a mouse, a rat, a dog, a cat, a horse, a cow, and the like.

The composition may be used alone, or in combination with surgery, hormone therapy, chemotherapy, and methods using a biological response modifier, for the prevention or treatment of fibrosis or scars.

Another aspect provides cell therapeutics for preventing or treating fibrosis or scars, including one or more selected from CMP cells and macrophages prepared by the above method of direct conversion.

The term “cell therapeutics” refers to therapeutics that use autologous, allogenic, and xenogenic cells in order to regenerate a function of tissue. The cell therapeutics, when including the macrophages induced by direct conversion as an active ingredient, may be utilized as cell therapeutics for preventing or treating fibrosis or scars.

The cell therapeutics may further include a pharmaceutically acceptable carrier. The pharmaceutically acceptable carrier may be, for example, saline solution, sterile water, Ringer's solution, buffered saline solution, dextrose solution, maltodextrin solution, glycerol, ethanol, human serum albumin (HSA), and a mixture of one or more thereof; and other common additives, such as an antioxidant, a buffer solution, and a bacteriostatic agent, may also be added as needed.

The cell therapeutics may be prepared as an injectable formulation.

Another aspect provides a composition for screening drugs for preventing or treating fibrosis or scars, the composition including one or more selected from CMP cells and macrophages prepared by the above method of direct conversion.

The composition for screening drugs may be utilized in screening drugs for treating fibrosis or scars, by confirming reactivity of one or more selected from CMP cells and macrophages prepared by the above method of direct conversion, in the presence or absence of a drug candidate for treating fibrosis or scars. For example, macrophages prepared by the method of direct conversion are cells that are involved in the healing or treatment of fibrosis or scars, and may be used to evaluate toxicity or drug efficacy with respect to drug candidate substances.

The assessment of toxicity may be carried out according to a known method of assessing toxicity commonly employed in the relevant field, such as assessing IC50 of the normal fibroblasts, the fibrosis-related fibroblasts or the macrophages induced by direct conversion, in the presence or absence of a drug candidate substance. Also, the evaluation of drug efficacy may be evaluated according to a known method that is capable of determining whether the macrophages induced by direct conversion have an effect of healing or treating fibrosis or scars, in the presence or absence of a drug candidate substance.

Another aspect provides a 3D printable biomaterial composition for fabricating artificial tissues for the treatment of fibrosis or scars, the composition including either the above composition for inducing direct conversion from somatic cells into CMP cells, or one or more selected from CMP cells and macrophages prepared by the above method of direct conversion.

The 3D printing technology is a printing technique for printing three-dimensional (3D) solid materials. The 3D printable biomaterial composition refers to a biocompatible polymer, natural polymer, biomolecule, biologically active material, and cell having the characteristics of biomimetic, mini-tissue and autonomous self-assembly.

The 3D printable biomaterial composition including either the composition for inducing direct conversion from somatic cells into CMP cells, or one or more selected from CMP cells and macrophages may be deposited layer by layer in a desired shape or pattern to thereby form artificial tissues for the treatment of fibrosis or scars, and thus may be widely utilized in the fields of regenerative medicine.

In the present application, duplicated information are omitted in the interest of simplicity, and the terms that are not otherwise defined in the present application may be understood to have the meaning as commonly used in the technical field to which the present disclosure belongs.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic diagram of preparation of macrophages using SOX2 transduction in human fibroblasts;

FIG. 2A shows flow cytometry results of analyzing the expression of mCitrine-SOX2 in neonatal fibroblasts which were transduced with mCitrine-SOX2 and cultured for 21 days;

FIG. 2B shows flow cytometry results of analyzing the expression of the blood cell marker CD45 and mCitirine-SOX2 in neonatal fibroblasts which were transduced with mCitrine-SOX2 and cultured for 21 days;

FIG. 2C shows flow cytometry results of analyzing the expression of the blood cell marker CD45 and mCitirine-SOX2 in neonatal fibroblasts which were transduced with mCitrine-SOX2 and cultured in blood cell maturation culture media;

FIG. 2D shows results of evaluating phagocytosis of macrophages which were differentiated from CD45-expressing cells after transduction of neonatal fibroblasts with mCitrine-SOX2;

FIG. 3 shows qRT-PCR results of analyzing the expression of pluripotency markers SOX2, NANOG, and OCT4, mesodermal lineage markers MIXL1 and BRACHY; and genes essential for hematopoiesis C/EBPα, PU.1, and SCL, in cells obtained by transducing neonatal fibroblasts with mCitirine-SOX2 and culturing the same for 21 days;

FIG. 4A shows flow cytometry results of analyzing the expression of mCitirine-SOX2 in adult fibroblasts which were transduced with mCitirine-SOX2 and cultured for 21 days;

FIG. 4B shows flow cytometry results of analyzing the expression of the blood cell marker CD45 and mCitirine-SOX2 in adult fibroblasts which were transduced with mCitirine-SOX2 and cultured for 21 days;

FIG. 4C shows flow cytometry results of analyzing the expression of the blood cell marker CD45 and mCitirine-SOX2 in adult fibroblasts which were transduced with mCitirine-SOX2 and cultured in blood cell maturation culture media;

FIG. 4D shows results of evaluating phagocytosis of macrophages which were differentiated from CD45-expressing cells in adult fibroblasts transduced with mCitrine-SOX2;

FIG. 5 shows qRT-PCR results of analyzing the expression of pluripotency markers SOX2, NANOG, and OCT4, mesodermal lineage markers MIXL1 and BRACHY; and genes essential for hematopoiesis C/EBPα, PU.1, and SCL, in cells which were obtained by transducing adult fibroblasts with mCitirine-SOX2 and culturing the same for 21 days;

FIG. 6 shows a schematic diagram of preparation of macrophages from fibroblasts by enhancing SOX2 expression with a chemical cocktail;

FIG. 7A shows flow cytometry results of analyzing the expression of the hematopoietic stem cell marker CD34 and the blood cell marker CD45 in cells which were obtained by adding a chemical cocktail to neonatal fibroblasts and culturing the same for 28 days;

FIG. 7B shows flow cytometry results of analyzing the expression of the blood cell marker CD45 and the CMP marker CD14 in cells which were obtained by adding a chemical cocktail to neonatal fibroblasts and culturing the same for 28 days;

FIG. 7C shows results of evaluating phagocytosis of macrophages which were differentiated from cells obtained by adding a chemical cocktail to neonatal fibroblasts and culturing the same for 28 days;

FIG. 8 shows qRT-PCR results of analyzing the expression of pluripotency markers SOX2, NANOG, and OCT4, mesodermal lineage markers MIXL1 and BRACHY; and genes essential for hematopoiesis C/EBPα, PU.1, and SCL, in cells obtained by adding a chemical cocktail to neonatal fibroblasts and culturing the same for 28 days;

FIG. 9A shows flow cytometry results of analyzing the expression of the hematopoietic stem cell marker CD34 and the blood cell marker CD45 in cells which were obtained by adding a chemical cocktail to adult fibroblasts and culturing the same for 28 days;

FIG. 9B shows flow cytometry results of analyzing the expression of the blood cell marker CD45 and the CMP marker CD14 in cells which were obtained by adding a chemical cocktail to adult fibroblasts and culturing the same for 28 days;

FIG. 9C shows results of evaluating phagocytosis of macrophages which were differentiated from cells obtained by adding a chemical cocktail to adult fibroblasts and culturing the same for 28 days;

FIG. 10 shows qRT-PCR results of analyzing the expression of pluripotency markers SOX2, NANOG, and OCT4, mesodermal lineage markers MIXL1 and BRACHY; and genes essential for hematopoiesis C/EBPα, PU.1, and SCL, in cells which were obtained by adding a chemical cocktail to adult fibroblasts and culturing the same for 28 days;

FIG. 11 shows qRT-PCR results of comparatively analyzing the expression of pluripotency markers SOX2, NANOG, and OCT4, mesodermal lineage markers MIXL1 and BRACHY; and genes essential for hematopoiesis C/EBPα, PU.1, and SCL, in cells (SOX2 OE) obtained by transducing neonatal fibroblasts (HDF-N) with SOX2, and cells (TβRlin) obtained by adding a chemical cocktail to neonatal fibroblasts (HDF-N);

FIG. 12 shows qRT-PCR results of comparatively analyzing the expression of pluripotency markers SOX2, NANOG, and OCT4; mesodermal lineage markers MIXL1 and BRACHY; and genes essential for hematopoiesis C/EBPα, PU.1, and SCL, in cells (SOX2 OE) obtained by transducing adult fibroblasts (HDF-A) with SOX2, and cells (TβRlin) obtained by adding a chemical cocktail to adult fibroblasts (HDF-A);

FIG. 13A shows qRT-PCR results of analyzing an expression level of SOX2 after inducing direct conversion by adding a chemical cocktail to fibroblasts. C (Control): fibroblasts not transduced with Tet-shSOX2; Tet-shSOX2 I and Tet-shSOX2 II: fibroblasts in which SOX2 expression is inhibited by tetracycline treatment in Tet-shSOX2 expressing cells;

FIG. 13B shows flow cytometry results of analyzing the expression of the blood cell marker CD45 after inducing direct conversion by adding a chemical cocktail to fibroblasts. Control: cells obtained by culturing fibroblasts not transduced with Tet-shSOX2, for 28 days without addition of a chemical cocktail; HDF+TβRlin: cells obtained by inducing direct conversion by adding a chemical cocktail to fibroblasts not transduced with Tet-shSOX2; Tet shSOX2-I+TβRlin and Tet shSOX2-II+TβRlin: cells obtained by inducing direct conversion by adding a chemical cocktail to fibroblasts in which SOX2 expression is inhibited by tetracycline treatment in Tet-shSOX2 expressing cells.

FIG. 14 shows results of flow cytometry analyses confirming the expression of the blood cell marker CD45 and the CMP marker CD14 in cells which were obtained after inducing direct conversion by adding a chemical cocktail to normal neonatal fibroblasts (HDF-N) and to neonatal fibroblasts in which wild type (WT), T204D (constitutively active form), or K232R (kinase dead form) of TGF-β type I receptor is overexpressed;

FIG. 15 shows qRT-PCR results of analyzing the expression of pluripotency markers SOX2, NANOG, and OCT4; mesodermal lineage markers MIXL1 and BRACHY; and genes essential for hematopoiesis C/EBPα, PU.1, and SCL in cells which were obtained after inducing direct conversion by adding a chemical cocktail to normal neonatal fibroblasts (HDF-N) and to neonatal fibroblasts in which WT, T204D, or K232R is overexpressed;

FIG. 16 shows results of flow cytometry analyses confirming the expression of the blood cell marker CD45 and the CMP marker CD14 in cells which were obtained after inducing direct conversion by adding a chemical cocktail to normal adult fibroblasts (HDF-A) and to adult fibroblasts in which WT, T204D, or K232R is overexpressed;

FIG. 17 shows qRT-PCR results of analyzing the expression of pluripotency markers SOX2, NANOG, and OCT4; mesodermal lineage markers MIXL1 and BRACHY; and genes essential for hematopoiesis C/EBPα, PU.1, and SCL, in cells which were obtained after inducing direct conversion by adding a chemical cocktail to normal adult fibroblasts (HDF-A) and to adult fibroblasts in which WT, T204D, or K232R is overexpressed;

FIG. 18 shows CD45 expression levels of cells obtained after culturing for 28 days with a different composition of chemical cocktail; and

FIG. 19 shows CD45 expression levels of cells obtained after culturing for 28 days with or without post-treatment with a GSK-3 inhibitor.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Herein below, the present disclosure will be described in greater detail with reference to embodiments. However, these embodiments are for illustrative purposes only and are not intended to be a limitation of the present disclosure.

<Materials and Experiment Methods>

1. Preparation of Cell Lines and Vectors

Human dermal fibroblasts derived from human skin were purchased from ThermoFisher (USA). Human neonatal dermal fibroblasts (HDF-N; neonatal-C0045C) and human adult dermal fibroblasts (HDF-A; adult-C0135C) were purchased, respectively.

The direct conversion (reprogramming or de-differentiation) factor to be transduced into the fibroblasts was SOX2, and lentiviral vectors (pLM-mCitrin-SOX2) were purchased from Addgene. The tetracycline (Tet)-induced knockdown vector (Tet-shSOX2) of SOX2 which is capable of selectively suppressing the expression of SOX2 by tetracycline treatment, was purchased from Addgene. Vectors (pcDNA3-ALK5 WT, pcDNA3-ALK5 T204D, pcDNA3-ALK5 K232R), which are capable of regulating the expression and activity of TGF-β type I receptor (TGFBR1, ALK5) were purchased from Addgene. In detail, pcDNA3-ALK5 WT is TGFBR1 wild type (WT) expression vector, pcDNA3-ALK5 T204D is constitutively active (CA) TGFBR1 T204D mutant expression vector, and pcDNA3-ALK5 K232R is kinase-dead (KD) TGFBR1 K232R mutant expression vector.

2. Evaluation of Functionality of Differentiated Macrophages

To examine whether the macrophages obtained after inducing macrophage differentiation for one week by SOX2 overexpression or a chemical cocktail have functionality, phagocytosis assay was conducted. In detail, 1 μm-sized latex beads (Sigma) were added to cell culture media and allowed to react for 60-90 minutes. The cells were washed with cold phosphate-buffered saline (PBS) and fixed using 4% paraformaldehyde solution. Cell morphology and phagocytosis of latex beads were observed under a microscope.

3. qRT-PCR Analyses for Reprogramming-Related Genes

To identify reprogramming characteristics of fibroblasts, the expression of pluripotency markers SOX2, NANOG, and OCT4; mesodermal lineage markers MIXL1 and BRACHY; and genes essential for hematopoiesis C/EBPα, PU.1, and SCL was confirmed by qRT-PCR.

The pluripotent markers were used to evaluate the stemness of stem cells upon reprogramming or direct conversion. The expression of the pluripotency marker in cells may be seen as an indication that pluripotency is acquired.

MXIL1 and BRACHY are factors that contribute to differentiation of mesodermal lineages and blood cells. These genes are reported to play a role in determining characteristics of mesodermal lineage and to develop their function. Blood cells are one of the cell types that can be differentiated from the mesoderm.

Further, C/EBPα is known to be a critical factor involved in differentiation/development of blood cells, and PU.1 is known to be an inducer of differentiation/development of monocytes/macrophages.

In detail, RNAs in the cells were isolated using Trizol, cDNA was synthesized using 1 μg of RNA, and primers in Table 1 were used to perform qRT-PCR. Analyses were achieved in a relative manner by taking the amount of the marker in normal fibroblasts (HDF-N or HDF-A) as 1.

TABLE 1

| Name of | | | |
|---|---|---|---|
| Gene | Direction | Sequence (5′→3′) | SEQ ID NO |
| SOX2 | Forward | GGGGGAAAGTAGTTTGCTGCCTCT | SEQ ID NO: 1 |
| | Reverse | CCTCCTCTGGCCGATCCTGC | SEQ ID NO: 2 |
| NANOG | Forward | CAGCCTCCAGCAGATGCAAGAACT | SEQ ID NO: 3 |
| | Reverse | TGAGGCCTTCTGCGTCACACC | SEQ ID NO: 4 |
| Oct4 | Forward | AGCAAAACCCGGAGGAGTCCC | SEQ ID NO: 5 |
| | Reverse | GCAGATGGTCGTTTGGCTGAATACC | SEQ ID NO: 6 |
| MIXL1 | Forward | AAACTGAGAAGTATCCTCTGCTAA | SEQ ID NO: 7 |
| | Reverse | TCTTCTGCAAGCCTCCCTAACACA | SEQ ID NO: 8 |
| BRACHY | Forward | ATGAGCCTCGAATCCACATAGT | SEQ ID NO: 9 |
| | Reverse | TCCTCGTTCTGATAAGCAGTCA | SEQ ID NO: 10 |
| C/EBPα | Forward | GAGGGACCGGAGTTATGACA | SEQ ID NO: 11 |
| | Reverse | TTCACATTGCACAAGGCACT | SEQ ID NO: 12 |
| PU.1 | Forward | GACAGGCAGCAAGAAGAAG | SEQ ID NO: 13 |
| | Reverse | TTGGACGAGAACTGGAAGG | SEQ ID NO: 14 |
| SCL | Forward | CAAAGTTGTGCGGCGTATCTT | SEQ ID NO: 15 |
| | Reverse | TCATTCTTGCTGAGCTTCTTGTC | SEQ ID NO: 16 |
| GAPDH | Forward | GGAGCGAGATCCCTCCAAAAT | SEQ ID NO: 17 |
| | Reverse | GGCTGTTGTCATACTTCTCATGG | SEQ ID NO: 18 |

4. FACS Analyses for Confirming Expression of Specific Proteins

To confirm direct conversion potential of fibroblasts to CMP cells, flow cytometry (FACS) analyses were performed. Using the antibodies listed below, expression levels of proteins including the hematopoietic stem cell (HSC) membrane marker CD34, the blood cell membrane marker CD45, and the common myeloid progenitor (CMP) membrane marker CD14, were confirmed. The antibodies used are as follows:
FITC conjugated mouse anti-human CD34 monoclonal antibodies, eBioscience
APC conjugated mouse anti-human CD45 monoclonal antibodies, eBioscience
APC-eFluoro780 conjugated mouse anti-human CD14 monoclonal antibodies, eBioscience

Cells were dissociated into single cells with accutase and then washed with 1% FBS/PBS solution. After treatment with Fc blocker (BD Bioscience) for 10 minutes to prevent non-specific antigen binding, the cells were blocked for 15 minutes using 1% FBS/PBS solution. An antigen-antibody reaction using a specific antibody was performed at room temperature for 1 hour. Normal fibroblasts reacted with APC Mouse IgG1 isotype were used as a control. Subsequent to the reaction, the cells were washed twice with cold PBS solution and analyzed by flow cytometry using CytoFLEX by Beckman, and data were analyzed using the CytExpert program.

### EXAMPLE 1

### Preparation of Macrophages from Fibroblasts Using SOX2 Overexpression

To prepare macrophages from fibroblasts, SOX2 was transduced into fibroblasts and overexpressed, such that the fibroblasts were converted into CMP cells. The cultured CMP cells were further differentiated into macrophages.

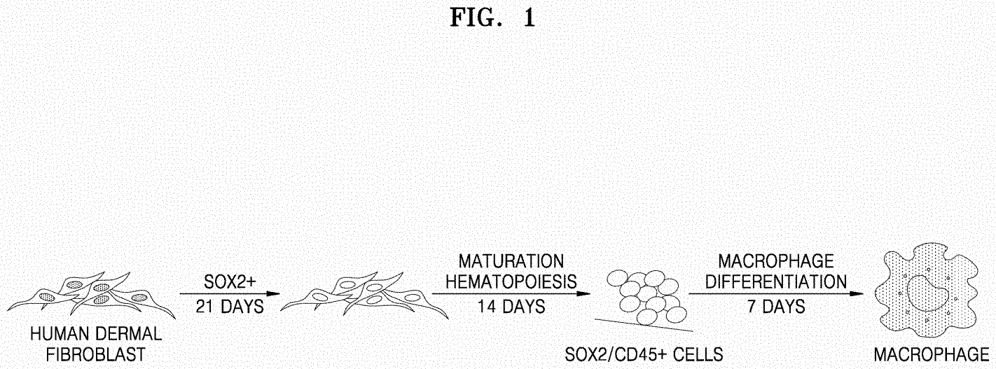

FIG. 1 is a diagram illustrating the preparation of macrophages from fibroblasts using SOX2 transduction.

(1) Reprogramming of Fibroblasts by SOX2 Overexpression

Fibroblasts were cultured on a geltrex- or matrigel-coated cell culture plate. Using lentiviral vector (pLM-mCitrin-SOX2) for SOX2 transduction, virus was generated from 293T cells, and media containing the generated virus were added to the fibroblasts. For fibroblast culture, Dulbecco's Modified Eagle's Medium (DMEM) supplemented with 10% fetal bovine serum (FBS), 1% penicillin streptomycin (P/S, penicillin), 1% glutamine (glutaMAX™-1), 1% non-essential amino acid (MEAA), and 0.055 mM β-mercaptoethanol were used.

After treatment with the virus as described above, the fibroblasts were cultured for 21 days with reprogramming media changes every 2-3 days. For the reprogramming media, KnockOut DMEM or DMEM/F-12 supplemented with 15% knockout Serum Replacement (KSR), 1% P/S, 1% glutamine (glutaMAX™-I), 1% NEAA, 30 ng/ml insulin-like growth factor II (IGFII), 20 ng/ml basic fibroblast growth factor (bFGF2), and 0.1 mM β-mercaptoethanol were used.

(2) Maturation of Blood Cells

The cells reprogrammed by SOX2 overexpression were dissociated into single cells using accutase and cultured in suspension using an ultra-low attachment culture dish. During the suspension-culture for 14 days, the cells were cultured with blood cell maturation culture media and the media was changes every 2-3 days. For the blood cell maturation culture media, media containing KnockOut DMEM supplemented with 20% bovine calf serum (BCS), 1% P/S, 1% glutamine (glutaMAX™-I), 1% NEAA, 1× Insulin-Transferrin-Selenium, 30 ng/ml thrombopoietin (TPO), 30 ng/ml stem cell factor (SCF), 20 ng/ml epidermal growth factor (EGF), and 0.1 mM β-mercaptoethanol were used.

(3) Differentiation into Macrophages

The CMP cells obtained by the above suspension-culture method were dissociated into single cells using accutase and cultured for one week in macrophage differentiation media. For the macrophage differentiation media, media containing RPMI1640 supplemented with 10% fetal bovine serum (FBS), 1% P/S, 10 ng/ml macrophage colony-stimulating factor (M-CSF), 10 ng/ml interleukin-4 (IL-4) and 0.055 mM β-mercaptoethanol were used.

(4) Characterization of Cells

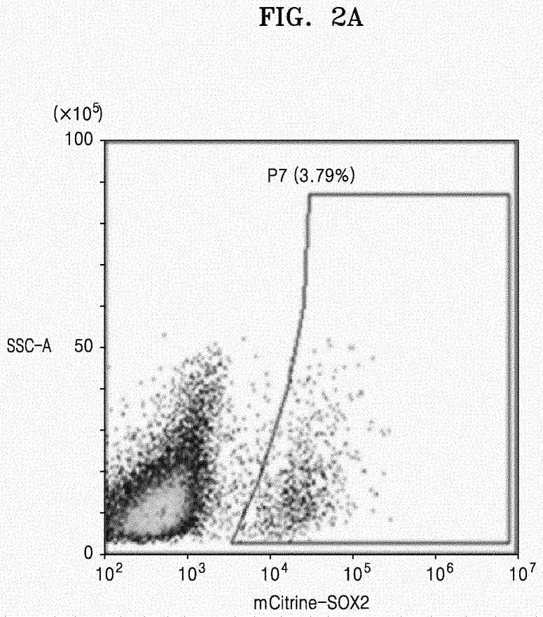

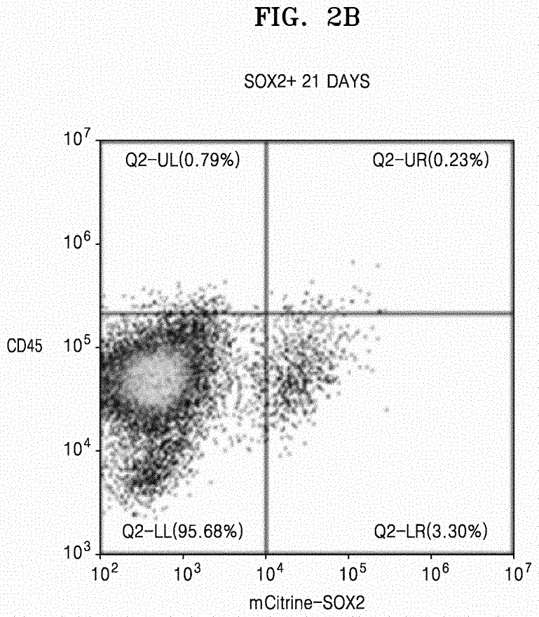

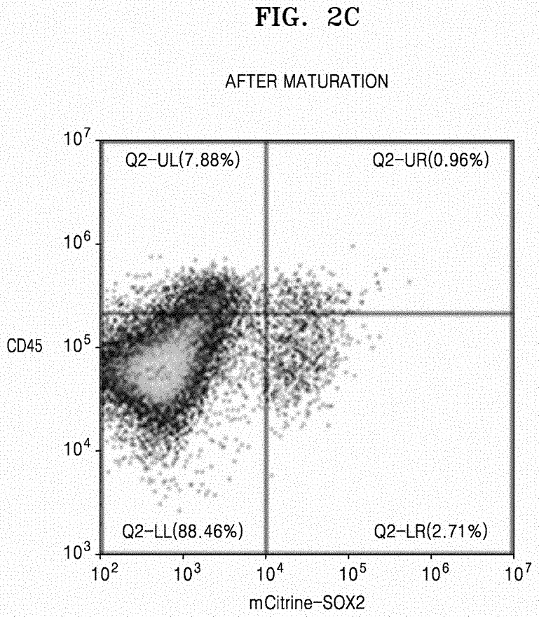

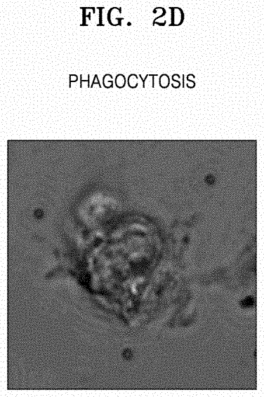

FIG. 2A shows flow cytometry results of analyzing mCitrine-SOX2 expression in neonatal fibroblasts that were transduced with mCitrine-SOX2 and cultured for 21 days. As shown in FIG. 2A, SOX2 fluorescence from mCitrine was detected, indicating successful overexpression of SOX2.

FIG. 2B shows flow cytometry results of analyzing the expression of the blood cell marker CD45 and mCitirine-SOX2 in neonatal fibroblasts that were transduced with mCitrine-SOX2 and cultured for 21 days. As shown in FIG. 2B, it could be confirmed that when mCitirine-SOX2 expression is induced, CD45 is slightly expressed.

FIG. 2C shows flow cytometry results of analyzing the expression of the blood cell marker CD45 and mCitirine-SOX2 in neonatal fibroblasts that were transduced with mCitrine-SOX2 and cultured in blood cell maturation culture media. As shown in FIG. 2C, after maturation, it could be confirmed that there is a slight increase in percentages of cells expressing CD45.

FIG. 2D shows results of evaluation of phagocytosis of macrophages that were differentiated from CD45-expressing cells in neonatal fibroblasts transduced with mCitrine-SOX2. As shown in FIG. 2D, the differentiated macrophages exhibit phagocytic ability.

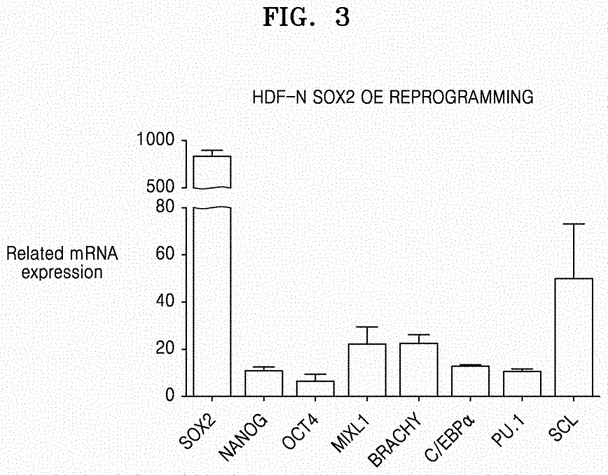

FIG. 3 shows qRT-PCR results of analyzing the expression of pluripotency markers SOX2, NANOG, and OCT4, mesodermal lineage markers MIXL1 and BRACHY; and genes essential for hematopoiesis C/EBPα, PU.1, and SCL, in cells obtained by transducing neonatal fibroblasts with mCitirine-SOX2 and culturing the same for 21 days.

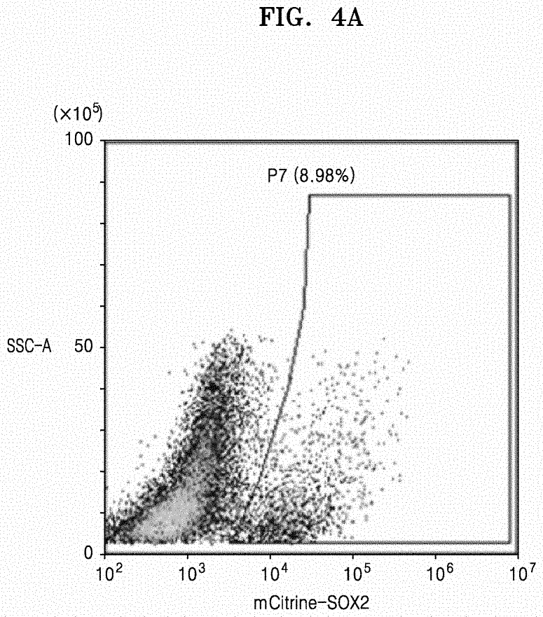

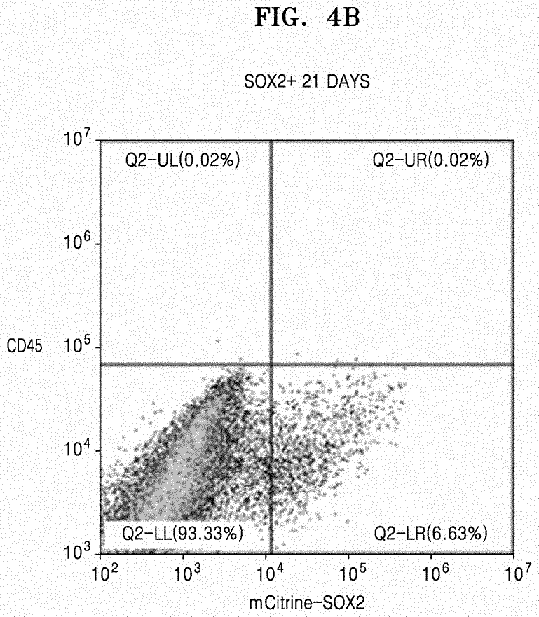

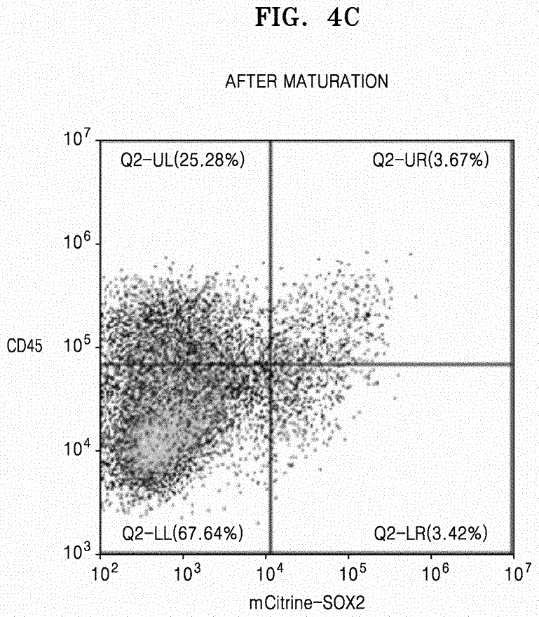

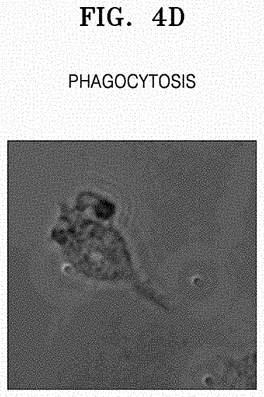

FIG. 4A shows flow cytometry results of analyzing the expression of mCitirine-SOX2 in adult fibroblasts that were transduced with mCitirine-SOX2 and cultured for 21 days. As shown in FIG. 4A, SOX2 fluorescence from mCitrine is detected, indicating a successful overexpression of SOX2.

FIG. 4B shows flow cytometry results of analyzing the expression of the blood cell marker CD45 and mCitirine-SOX2 in adult fibroblasts that were transduced with mCitirine-SOX2 and cultured for 21 days. As shown in FIG. 4B, it could be confirmed that when mCitirine-SOX2 expression is induced, CD45 is slightly expressed.

FIG. 4C shows flow cytometry results of analyzing the expression of the blood cell marker CD45 and mCitirine-SOX2 in adult fibroblasts that were transduced with mCitirine-SOX2 and cultured in blood cell maturation media. As shown in FIG. 4C, it could be confirmed that in a similar fashion as in neonatal fibroblasts, after maturation, CD45 expression level is increased, inducing direct conversion. Conversion efficiency is higher than that observed in neonatal fibroblasts.

FIG. 4D shows results of evaluating phagocytosis of macrophages that were differentiated from CD45-expressing cells in adult fibroblasts transduced with mCitrine-SOX2. As shown in FIG. 4D, the differentiated macrophages exhibit phagocytic ability.

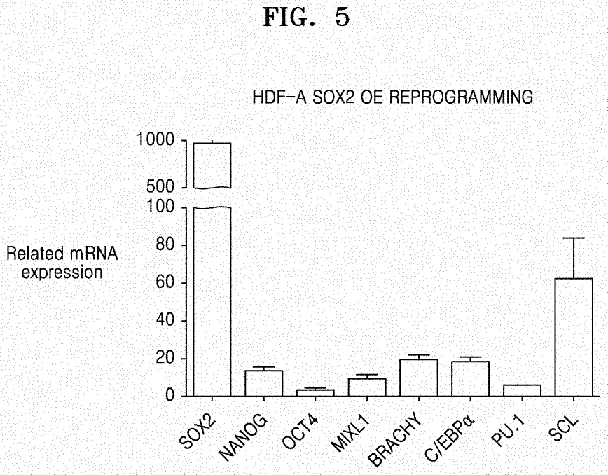

FIG. 5 shows qRT-PCR results of analyzing the expression of pluripotency markers SOX2, NANOG, and OCT4; mesodermal lineage markers MIXL1 and BRACHY; and genes essential for hematopoiesis C/EBPα, PU.1, and SCL, in cells obtained by transducing adult fibroblasts with mCitirine-SOX2 and culturing the same for 21 days.

Taking FIG. 2 to FIG. 5 together, it could be confirmed that the transduction of SOX2 alone results in the overexpression of factors related to blood cell direct conversion. Although the expression of the haematopoietic stem cell marker CD34 was not observed (data not shown), the expression of the blood cell marker CD45 was confirmed, indicating the possibility of reprogramming the fibroblasts to CMP cells. Also, it could be confirmed that the cells obtained by SOX2 overexpression are able to differentiate into macrophages with phagocytotic activity. In this regard, related art document (J. Pulecio et al., “Conversion of human fibroblasts into monocyte-like progenitor cells”, Stem Cells. 2014 Nov;32(11):2923-2938. doi: 10.1002/stem.1800.) reports that when inducing direct conversion in normal fibroblasts by treatment with a combination of SOX2 and miR-125b together, the conversion efficiency was increased. However, SOX2 introduction alone results in a low conversion efficiency.

### EXAMPLE 2

### Preparation of Macrophages from Fibroblasts Using Chemical Cocktail

To prepare macrophages from fibroblasts, a compound for enhancing SOX2 expression was added to the fibroblasts, and then the treated cells were matured into CMP cells. The cultured CMP cells were differentiated into macrophages.

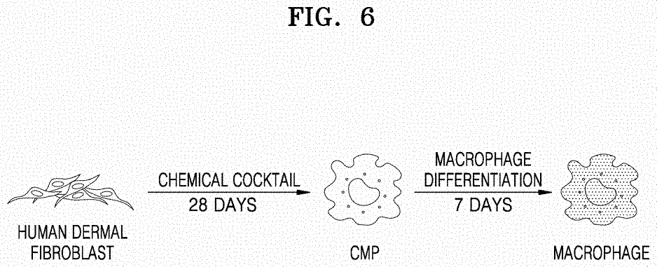

FIG. 6 is a diagram illustrating the preparation of fibroblasts into macrophages by enhancing SOX2 expression using a chemical cocktail.

(1) Pretreatment of Fibroblasts

In a process prior to inducing the direct conversion of fibroblasts into CMP, the fibroblasts were pretreated. Pretreatment of fibroblasts is an optional process that may enhance the efficiency of direct conversion of fibroblasts into CMP cells.

Fibroblasts were cultured on a geltrex- or matrigel-coated cell culture plate. For pretreatment culture media, media containing DMEM supplemented with 10% FBS, 1% P/S, 1% glutamine (glutaMAX™-I), 1% NEAA, 50 μg/ml vitamin C (VitC), and 0.055 mM β-mercaptoethanol were used. 24 hours after culture initiation, valproic acid (VPA, 0.5 mM) was added to the media and cultured for 24 hours.

(2) Direct Conversion of Fibroblasts to CMP Cells by Addition of Chemical Cocktail

The pretreated cells were subjected to first culturing in reprogramming media supplemented with a chemical cocktail. In the first culturing, the media was changed every 2-3 days, and cells were subcultured once every weeks for 14 days in total. The chemical cocktail includes a TGF-β receptor inhibitor (616452), VPA, and VitC. VitC, which is an antioxidant, was used to increase conversion efficiency, but is not a substance that is necessary for direct conversion. The detailed media composition is represented in Table 2 below.

TABLE 2

| | Reprogramming Media Composition |
|---|---|
| Classification | Supplemented with Chemical Cocktail |
| Base | KnockOut DMEM |
| Reprogramming | 15% KSR (Knockout Serum Replacement) |
| Media | 1% penicillin streptomycin (P/S, penicillin) |
| | 1% glutamine (glutaMAXTM-I) |
| | 1% NEAA (non-essential amino acids) |
| | 30 ng/ml IGFII (insulin-like growth factor II) |
| | 20 ng/ml bFGF2 (basic fibroblast growth factor 2) |
| | 0.1 mM β-mercaptoethanol |
| Chemical Cocktail | TGFβRI inhibitor (616452, 10 μM) |
| | 0.5 mM VPA |
| | 100 μg/ml VitC |

Subsequently, in second culturing, cells were cultured in media containing the above media composition with additionally supplement of a glycogen synthase kinase 3 (GSK-3) inhibitor (CHIR99021). The concentration of the GSK-3 inhibitor in the media was 3 μM. The cells were subcultured once every weeks for 14 days in total, while the media was changed every 2-3 days.

(3) Differentiation into Macrophages

The CMP cells obtained by direct conversion above were cultured in macrophage differentiation media for one week. For the macrophage differentiation media, media containing RPMI1640 supplemented with 10% fetal bovine serum (FBS), 1% P/S, 10 ng/ml macrophage colony-stimulating factor (M-CSF), 10 ng/ml interleukin-4 (IL4), and 0.055 mM β-mercaptoethanol were used.

(4) Characterization of Cells

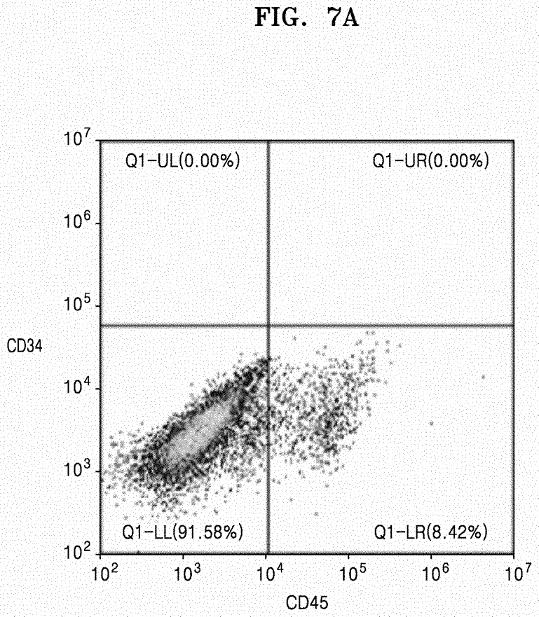

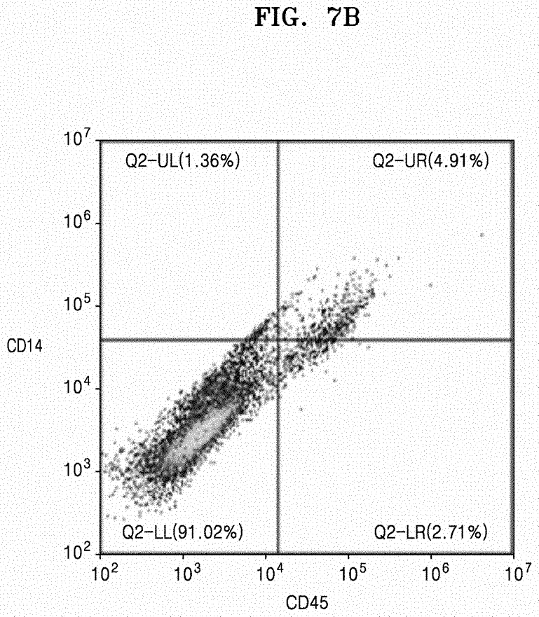

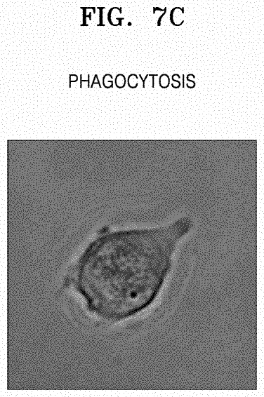

FIG. 7A shows flow cytometry results of analyzing the expression of the hematopoietic stem cell marker CD34 and the blood cell marker CD45 in cells that were obtained by adding a chemical cocktail to neonatal fibroblasts and culturing the same for 28 days. As shown in FIG. 7A, CD34 was not expressed, indicating that the cultured cells were not hematopoietic stem cells. Meanwhile, the number of cells expressing CD45 was increased.

FIG. 7B shows flow cytometry results of analyzing the expression of the blood cell marker CD45 and the CMP marker CD14 in cells that were obtained by adding a chemical cocktail to neonatal fibroblasts and culturing the same for 28 days. As shown in FIG. 7B, the expression of CD45 and CD14 was confirmed, indicating that the fibroblasts were differentiated into CMP lineage cells, not into hematopoietic stem cells.

FIG. 7C shows results of evaluating phagocytosis of macrophages differentiated from cells that were obtained by adding a chemical cocktail to neonatal fibroblasts and culturing the same for 28 days. As shown in FIG. 7C, the differentiated macrophages exhibited phagocytic activity.

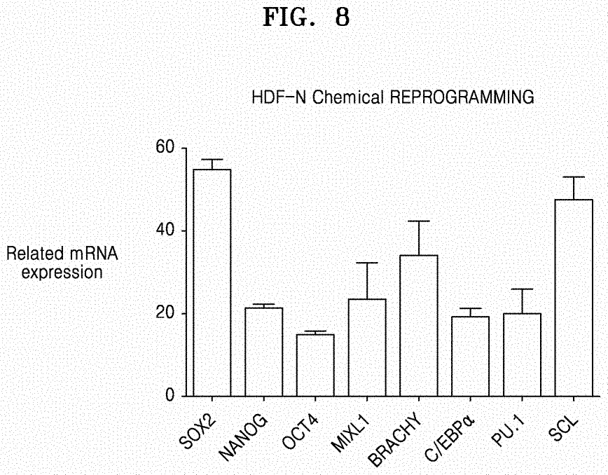

FIG. 8 shows qRT-PCR results of analyzing the expression of pluripotency markers SOX2, NANOG, and OCT4; mesodermal lineage markers MIXL1 and BRACHY; and genes essential for hematopoiesis C/EBPα, PU.1, and SCL, in cells obtained by adding a chemical cocktail to neonatal fibroblasts and culturing the same for 28 days.

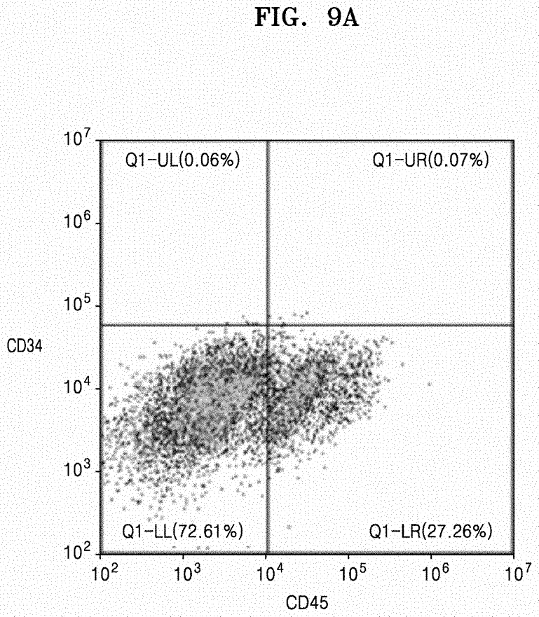

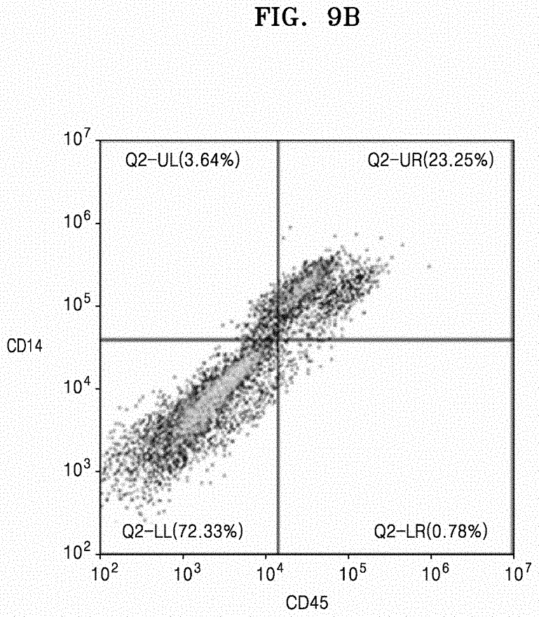

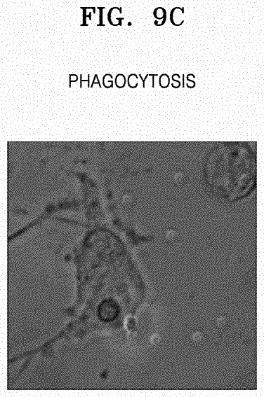

FIG. 9A shows flow cytometry results of analyzing the expression of the hematopoietic stem cell marker CD34 and the blood cell marker CD45 in cells that were obtained by adding a chemical cocktail to adult fibroblasts and culturing the same for 28 days. As shown in FIG. 9A, in a similar fashion as observed with the neonatal fibroblasts, as CD34 was not expressed, the number of cells expressing CD45 was increased; and since CD34 was not expressed in these cells, it could be confirmed that the cultured cells were not hematopoietic stem cells. Meanwhile, the percentage of cells expressing CD45 was noticeably increased compared with the neonatal fibroblasts.

FIG. 9B shows flow cytometry results of analyzing the expression of the blood cell marker CD45 and the CMP marker CD14 in cells that were obtained by adding a chemical cocktail to adult fibroblasts and culturing the same for 28 days. As shown in FIG. 9B, the percentages of cells expressing CD45 and CD14 were noticeably increased compared with the neonatal fibroblasts. In other words, it could be confirmed that there is an increase in conversion efficiency toward CMP lineage cells in human adult fibroblasts, compared with that observed in neonatal fibroblasts.

FIG. 9C shows results of evaluating phagocytosis of macrophages differentiated from cells that were obtained by adding a chemical cocktail to adult fibroblasts and culturing the same for 28 days. As shown in FIG. 9C, the differentiated macrophages exhibited phagocytic activity.

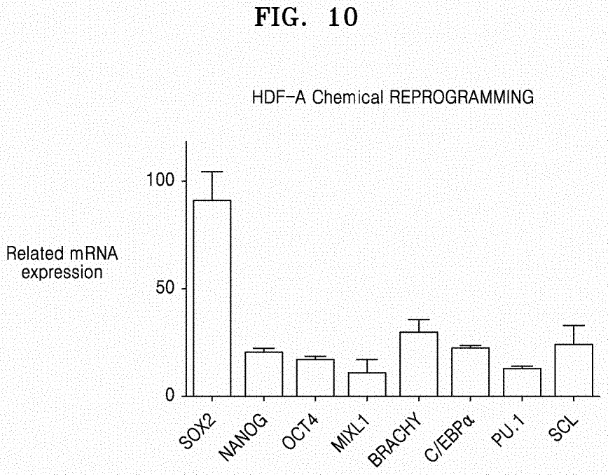

FIG. 10 shows qRT-PCR results of analyzing the expression of pluripotency markers SOX2, NANOG, and OCT4, mesodermal markers MIXL1 and BRACHY; and genes essential for hematopoiesis C/EBPα, PU.1, and SCL in cells that were obtained by adding a chemical cocktail to adult fibroblasts and culturing the same for 28 days.

Taking FIG. 7 to FIG. 10 together, the cells obtained by the direct conversion method using the chemical cocktail showed a negligible level of CD34 expression and demonstrated the expression of CD45 and CD14. Thus, it could be confirmed that the cells were differentiated into CMP lineage cells, not into hematopoietic stem cells. Further, it could be confirmed that the cells obtained by direct conversion using the chemical cocktail were able to differentiate into macrophages with phagocytic activity. These results could be also confirmed by analyzing related genes.

Further, since the CMP cells obtained by the direct conversion method using the chemical cocktail as described in Example 2 exhibited a higher level of CD45 expression compared with that observed in CMP cells obtained by the direct conversion method using SOX2 overexpression as described in Example 1, it could be confirmed that the method in Example 2 has superior conversion efficiency.

### EXPERIMENTAL EXAMPLE 1

### Comparison Between SOX2 Overexpression-Based Direct Conversion and Direct Conversion Using Chemical Cocktail

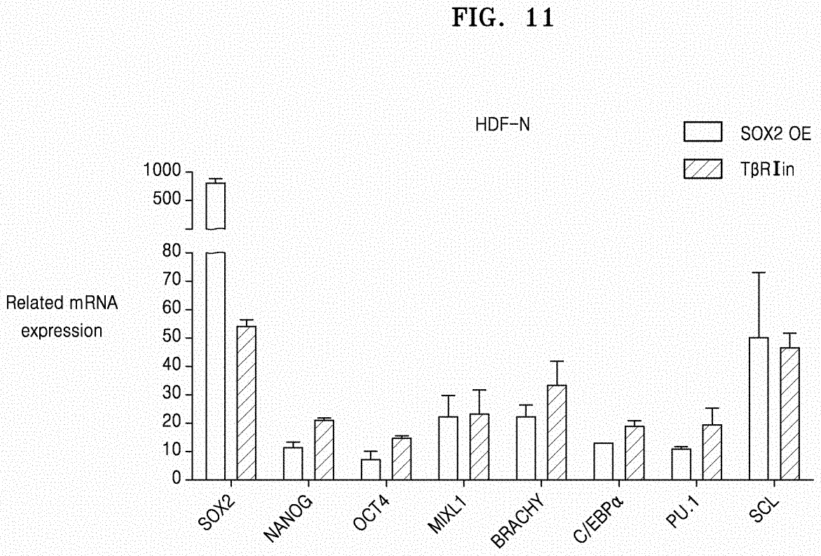

FIG. 11 shows qRT-PCR results of comparatively analyzing the expression of pluripotency markers SOX2, NANOG, and OCT4, mesodermal lineage markers MIXL1 and BRACHY; and genes essential for hematopoiesis C/EBPα, PU.1, and SCL, in cells (SOX2 OE) that were obtained by transducing neonatal fibroblasts (HDF-N) with SOX2; and in cells (TβRlin) that were obtained by adding a chemical cocktail to neonatal fibroblasts (HDF-N).

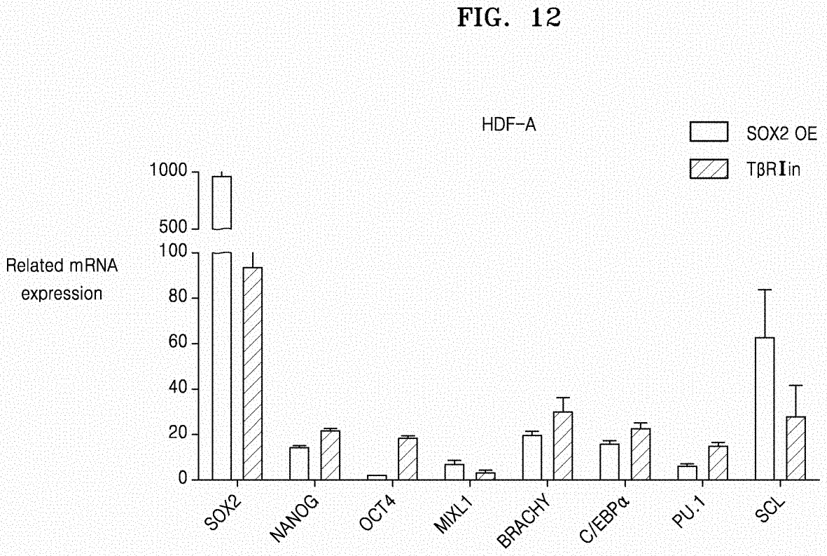

FIG. 12 shows qRT-PCR results of comparatively analyzing the expression of pluripotency markers SOX2, NANOG, and OCT4, mesodermal lineage markers MIXL1 and BRACHY; and genes essential for hematopoiesis C/EBPα, PU.1, and SCL, in cells (SOX2 OE) that were obtained by transducing adult fibroblasts (HDF-A) with SOX2; and in cells (TβRlin) that were obtained by adding a chemical cocktail to adult fibroblasts (HDF-A).

Taking FIG. 11 to FIG. 12 together, in all cells obtained by direct conversion, either by SOX2 overexpression or using the chemical cocktail, the expression of pluripotency markers SOX2, NANOG, and OCT4, mesodermal lineage markers MIXL1 and BRACHY; and genes essential for hematopoiesis C/EBPα, PU.1, and SCL, was enhanced in a similar fashion. However, it could be confirmed that there is a further increase in the expression of NANOG and OCT4 in the direct conversion conditions using the chemical cocktail. Further, it could be confirmed that there is a further increase in the expression of mesodermal lineage markers MIXL1 and Brachy, and markers essential for hematopoiesis C/EBPα and PU.1 in the direct conversion conditions using the chemical cocktail.

Accordingly, it could be confirmed that the direct conversion method using the chemical cocktail has a superior conversion efficiency compared to that of the direct conversion method using SOX2 overexpression.

### EXPERIMENTAL EXAMPLE 2

### Confirmation of SOX2 Dependency of Direct Conversion Using Chemical Cocktail

To understand the correlation of whether SOX2 enhancement by a chemical cocktail has an effect on conversion efficiency, the following experiments were prepared.

By transducting Tet-shSOX2 vector using lentiviral delivery, two batches of Tet-shSOX2 expressing cell lines; SOX2-I and SOX2-II, were established. Each cell lines were treated with doxycycline to suppress SOX2 expression. The cells were supplemented with a chemical cocktail in an identical manner as shown in Example 2 (1) and (2) and cultured for 28 days to induce direct conversion.

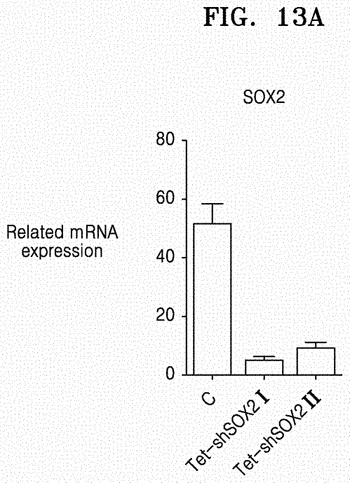

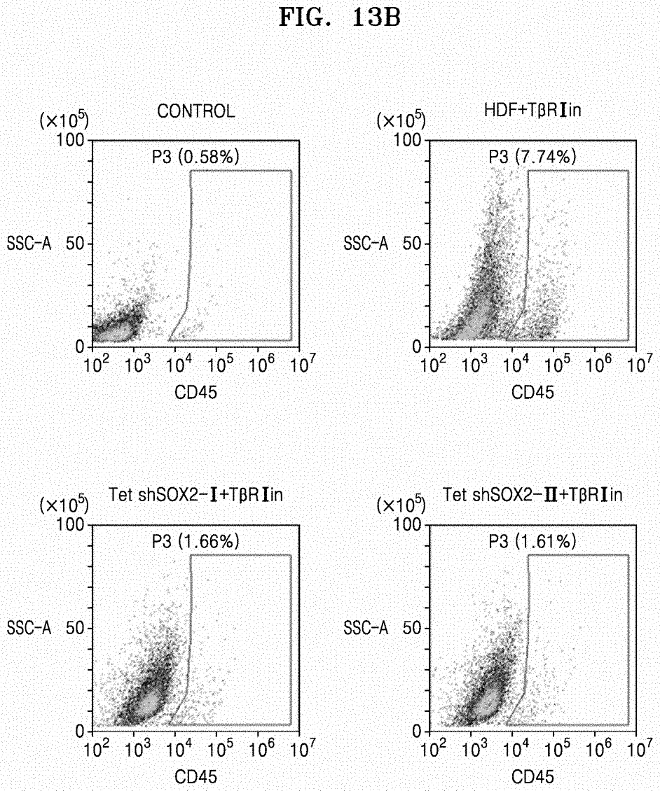

FIG. 13A shows qRT-PCR results of analyzing an expression level of SOX2 after inducing direct conversion by adding a chemical cocktail to fibroblasts.

FIG. 13B shows flow cytometry results of analyzing the expression of the blood cell marker CD45 after inducing direct conversion by adding a chemical cocktail to fibroblasts.

As shown in FIGS. 13A and 13B, it could be confirmed that direct conversion from fibroblasts into CMP lineage cells using the chemical cocktail occurs in a manner dependent on SOX2 expression enhancement.

### EXPERIMENTAL EXAMPLE 3

### Correlation Between SOX2 Enhancement Effect by Chemical Cocktail and TGF-B Receptor Type I Activity Status, and Identification of Their Effect on Conversion Efficiency

To confirm that SOX2 enhancement effect by a chemical cocktail is mediated from inhibition of TGF-β type I receptor, experiments were performed as follows.

By transducing vectors pcDNA3-ALK5 WT, pcDNA3-ALK5 T204D, or pcDNA3-ALK5 K232R into fibroblasts, cell lines expressing wild type (WT) of TGFβRI, constitutively active mutant (CA, T204D), and inactive mutant (KD, K232R) were established. Each of the cell lines was supplemented with a chemical cocktail in a manner identical to the method shown in Example 2 (1) and (2), and was cultured for 28 days to induce direct conversion.

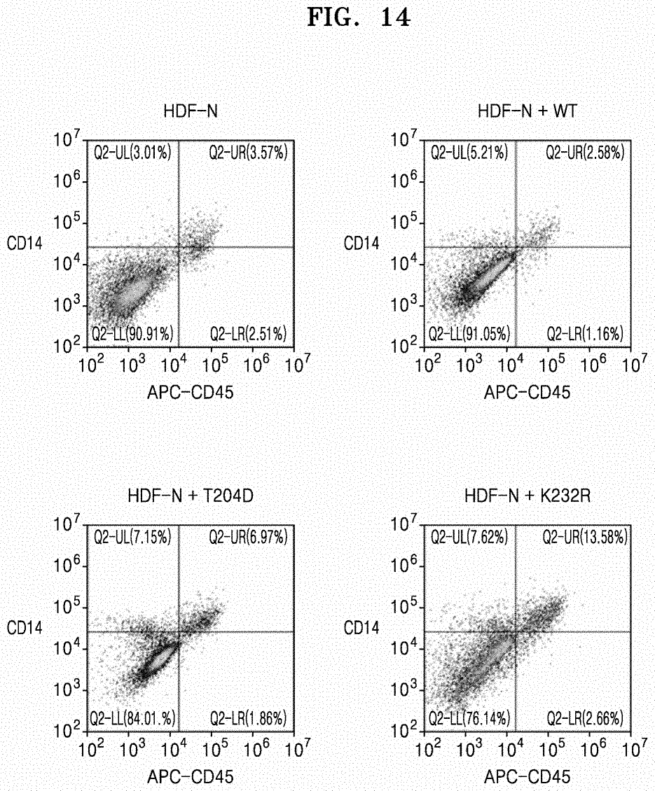

FIG. 14 shows flow cytometry results of analyzing the expression of the blood cell marker CD45 and the CMP marker CD14 in cells that were obtained after inducing direct conversion by adding a chemical cocktail to normal neonatal fibroblasts (HDF-N) and to neonatal fibroblasts in which WT, T204D, or K232R is overexpressed.

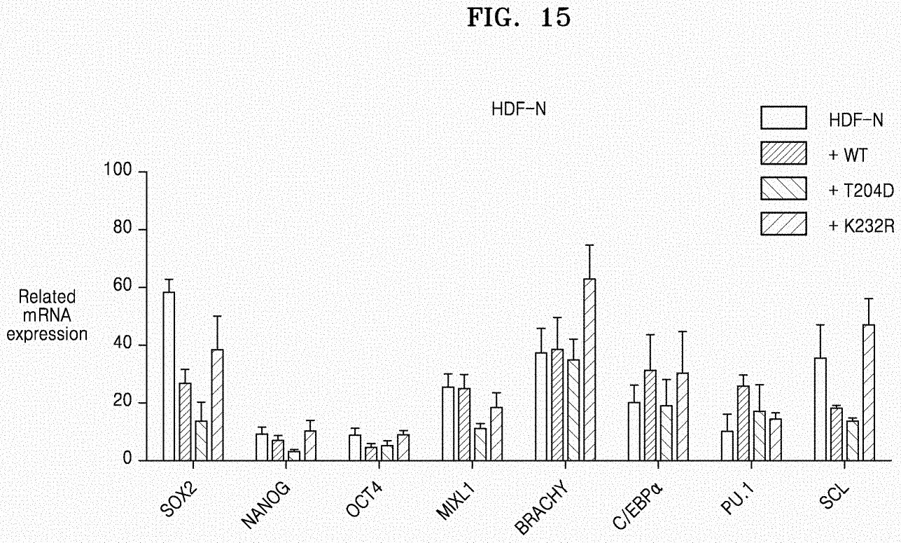

FIG. 15 shows qRT-PCR results of analyzing the expression of pluripotency markers SOX2, NANOG, and OCT4; mesodermal lineage markers MIXL1 and BRACHY; and genes essential for hematopoiesis C/EBPα, PU.1, and SCL in cells that were obtained after inducing direct conversion by adding a chemical cocktail to normal neonatal fibroblasts (HDF-N) and to neonatal fibroblasts in which WT, T204D, or K232R is overexpressed.

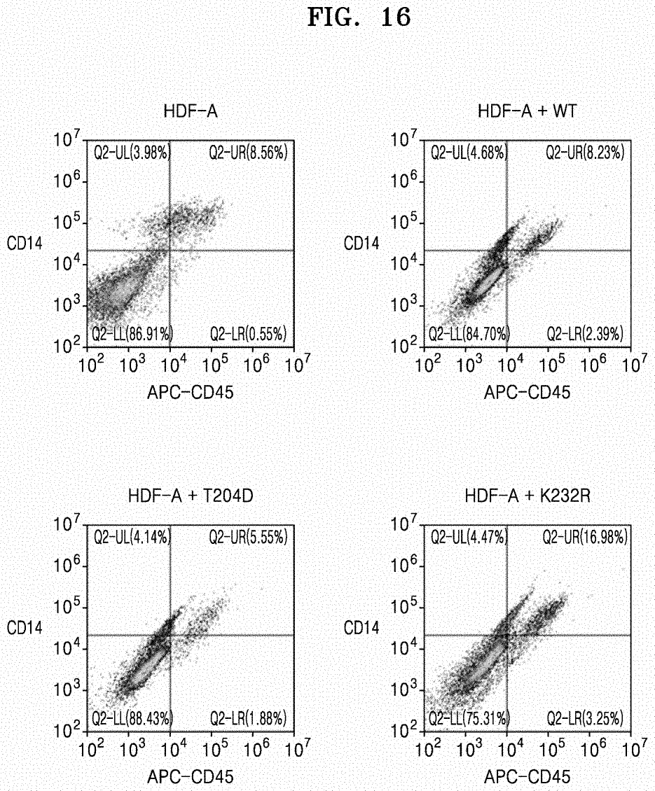

FIG. 16 shows results of flow cytometry analyses confirming the expression of the blood cell marker CD45 and the CMP marker CD14 in cells that were obtained after inducing direct conversion by adding a chemical cocktail to normal adult fibroblasts (HDF-A) and to adult fibroblasts in which WT, T204D, or K232R is overexpressed.

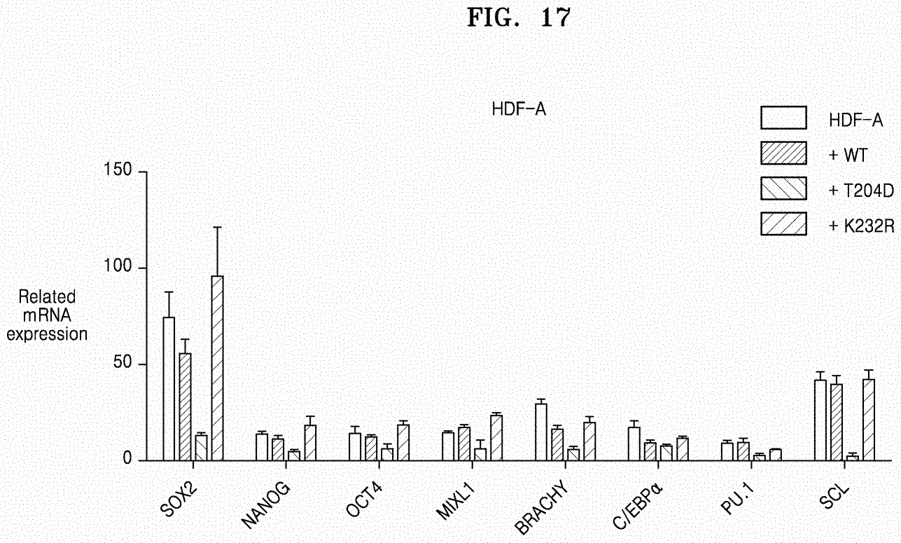

FIG. 17 shows qRT-PCR results of analyzing the expression of pluripotency markers SOX2, NANOG, and OCT4; mesodermal lineage markers MIXL1 and BRACHY, and genes essential for hematopoiesis C/EBPα, PU.1, and SCL, in cells that were obtained after inducing direct conversion by adding a chemical cocktail to normal adult fibroblasts (HDF-A) and to adult fibroblasts in which WT, T204D, or K232R is overexpressed.

Taking FIG. 14 to FIG. 17 together, as there is a noticeable increase in the expression of CD45 and CD14 proteins in the K232R cells in which suppressed TGF-β receptor type I function in the neonatal fibroblasts and the adult fibroblasts, the effect of the chemical cocktail on direct conversion could be confirmed. Further, it could be confirmed that there is a further increase in the expression of BRACHY and SCL in K232T cells after inducing direct conversion using a chemical cocktail, compared with that observed in normal neonatal fibroblasts. Further, it could be confirmed that there is a further increase in SOX2 expression in KD cells after inducing direct conversion using the chemical cocktail, compared with that observed in normal adult fibroblasts.

Therefore, it was confirmed that fibroblasts could be directly converted into CMP cells by inhibiting TGF-β activity, thereby increasing SOX2 expression.

### EXAMPLE 3

### Direct conversion From Fibroblasts to CMP Cells With the Addition of a Compound From a Chemical Cocktail

To determine the effect of addition of individual compound from the chemical cocktail, direct conversion from fibroblasts to CMP cells was induced by adding various combinations of compounds.

In detail, neonatal fibroblasts (HDF-N) were cultured in reprogramming media containing chemical compositions shown in Table 3. The reprogramming media were composed of base reprogramming media and a chemical composition, and the composition of the base reprogramming media is the same as that of the base reprogramming media described in Table 2 in Example 2. With media changes every 2-3 days and sub-culturing performed once a week, cells were cultured over 28 days in total.

Cells cultured with the base reprogramming media without addition of the compounds were used as a negative control group (Cnt).

CD45 expression level in the cells obtained after culturing for 28 day was used to determine direct conversion capability. Relative CD45-expressing cells (%) in each experiment group in comparison with the negative control group were shown in Table 3.

TABLE 3

| | | CD45-expressing cell |
|---|---|---|
| | | (%) compared to |
| Experiment | Chemical | negative control |
| groups | compositions | group (Cnt) |
| Example 3-1 | 10 μM TGFβRI inhibitor (616452) | 6.36% |
| Example 3-2 | 10 μM TGFβRI inhibitor | 3.26% |
| | (616452) + 100 μg/ml VitC | |
| Example 3-3 | 10 μM TGFβRI inhibitor | 9.13% |
| | (616452) + 0.5 mM VPA | |
| Example 3-4 | 10 μM TGFβRI inhibitor | 15% |
| | (616452) + 0.5 mM VPA + | |
| | 100 μg/ml VitC | |

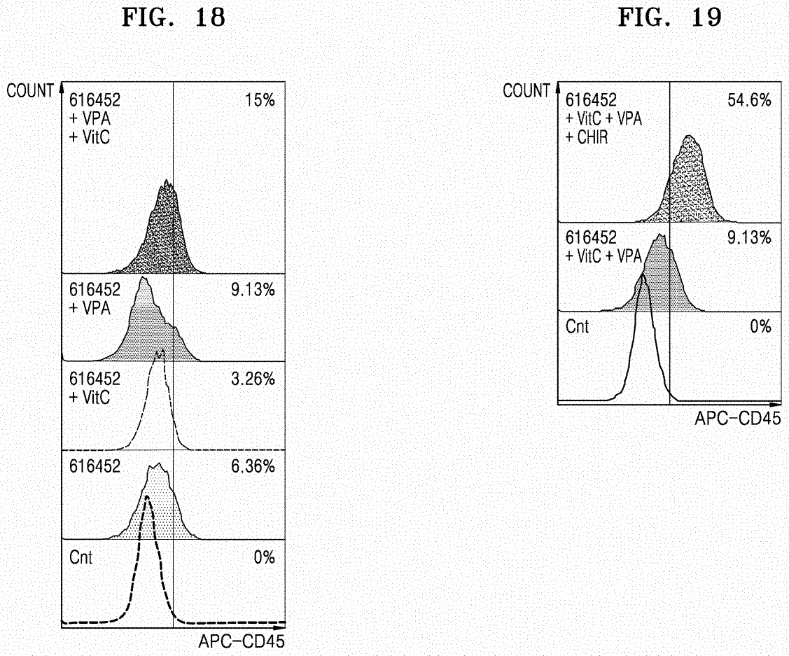

FIG. 18 shows CD45 expression levels of cells obtained after culturing for 28 days according to chemical compositions.

As shown in Table 3 and FIG. 18, it was found that the use of TGF-β receptor inhibitor alone led to an increase in the expression of the blood cell marker CD45. Accordingly, it could be confirmed that from the chemical cocktail, the TGF-β receptor inhibitor alone can be used for the direct conversion of somatic cells to CMP cells.

Also, it was found that using the TGF-β receptor inhibitor and a HDAC inhibitor (e.g., VPA) together leads to an increase in conversion efficiency. It was also found that using the TGF-β receptor inhibitor and an antioxidant (e.g., VitC) together leads to an increase in cell viability. It was also found that using the TGF-β receptor inhibitor, the HDAC inhibitor, and the antioxidant all together leads to not only an increase in cell viability but also an increase in direct conversion capability.

### EXAMPLE 4

### Effect of Post-treatment of GSK-3 Inhibitor on the Conversion Efficiency

Experiments were performed to determine the effect of post-treatment of GSK-3 inhibitor on the conversion efficiency.

In detail, neonatal fibroblasts (HDF-N) were cultured in reprogramming media containing chemical compositions shown in Table 4. The reprogramming media were composed of base reprogramming media and a chemical composition, and the composition of the base reprogramming media was the same as that of the base reprogramming media described in Table 2 in Example 2.

Cells were cultured for 28 days using a combination of a TGF-β receptor inhibitor, an HDAC inhibitor, and an antioxidant in Example 4-1. Cells in Example 4-2 were obtained by performing a first culture for 14 days using a combination of a TGF-β receptor inhibitor, an HDAC inhibitor, and an antioxidant, and then a second culture for 14 days using a combination of a TGF-β receptor inhibitor, an HDAC inhibitor, an antioxidant, and a GSK-3 inhibitor. In Examples 4-1 and 4-2, the media were changed once every 2-3 days, and the cells were sub-cultured once a week.

Cells cultured using the base reprogramming media without addition of the compounds were used as a negative control group (Cnt).

CD45 expression level in the cells obtained after culturing for 28 day was used to determine direct conversion capability. CD45-expressing cells in each experiment group in comparison with the negative control group were shown in Table 4.

TABLE 4

| | | CD45-expressing cell |
|---|---|---|
| | | (%) compared to |
| | Chemical | negative control group |
| Examples | Compositions | (Cnt) |
| Example 4-1 | 10 μM TGFβRI inhibitor (616452) + | 9.13% |
| (cultured for | 0.5 mM VPA + 100 μg/ml VitC | |
| 28 days) | | |
| Example 4-2 | First culture (14 days): 10 μm | 54.6% |
| (cultured for | TGFβRI inhibitor (616452) + 0.5 mM | |
| 28 days) | VPA + 100 μg/ml VitC | |
| | Second culture (14 days): 10 μM | |
| | TGFβRI inhibitor (616452) + 0.5 mM | |
| | VPA + 100 μg/ml VitC + 3 μM | |
| | GSK-3 inhibitor (CHIR99021) | |

FIG. 19 shows CD45 expression levels of cells obtained after culturing for 28 days, with or without post-treatment with a GSK-3 inhibitor.

As shown in Table 4 and FIG. 19, it was found that cells post-treated with the GSK-3 inhibitor showed an increased conversion efficiency compared to the cells treated with only a TGF-β receptor inhibitor, an HDAC inhibitor, and an antioxidant.

According to a composition for inducing direct conversion of somatic cells into CMP cells, according to one aspect, the composition including a chemical cocktail, it is possible to prepare CMP cells and macrophages with a higher yield within a shorter period of time, compared with existing methods using gene transduction. Further, by using drugs that are actually clinically applied, it is possible to directly convert somatic cells into CMP cells and macrophages without genetic manipulation or modification. Accordingly, the composition, or CMP cells and macrophages prepared using the composition may be used for preventing or treating diseases associated with fibroblasts, for example, chronic-refractory conditions, such as fibrosis and scars.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A composition for inducing direct conversion from somatic cells into common myeloid progenitor (CMP) cells, the composition comprising a TGF-β receptor inhibitor.

2. The composition of claim 1, further comprising a histone deacetylase (HDAC) inhibitor, glycogen synthase kinase 3 (GSK-3) inhibitor, or a combination thereof.

3. The composition of claim 1, wherein the TGF-β receptor inhibitor is 2-[3-(6-methylpyridin-2-yl)-1H-pyrazol-4-y]-1,5-naphthyridine (616452), SB431542, galunisertib (LY2157299), LY3200882, vactosertib (TEW-7197), PF-06952229, or a combination of two or more thereof.

4. The composition of claim 2, wherein the HDAC inhibitor is a valproate, Trichostatin A, phenylbutyrate, sodium butyrate, suberoylanilide hydroxamic acid (SAHA), suberohydroxamic acid (SBHA), or a combination of two or more thereof.

5. The composition of claim 4, wherein the valproate is valproic acid (VPA), sodium valproate, divalproex sodium, or a combination of two or more thereof.

6. The composition of claim 2, wherein the GSK-3 inhibitor is 6-((2-((4-(2,4-dichlorophenyl)-5-(4-methyl-1H-imidazol-2-yl)pyrimidin-2-y1)amino)ethyl)amino)nicotinonitrile (CHIR99021), TD114-2, SB216763, SB415286, or a combination of two or more thereof.

7. The composition of claim 1, further comprising an antioxidant.

8. The composition of claim 7, wherein the antioxidant is ascorbic acid, resveratrol, acetylcysteine, ethylbisiminomethylguaiacol manganese chloride (EUK-134), an NADPH oxidase inhibitor, or a combination of two or more thereof.

9. The composition of claim 1, wherein the somatic cells are one or more selected from fibroblasts, adipose stromal cells, epithelial cells, muscle cells, oral epithelial cells, somatic cells extracted from urine, blood cells, hair follicle stem cells, neural stem cells, hematopoietic stem cells, and mesenchymal stem cells.

10. The composition of claim 1, wherein the CMP cells are able to differentiate into myeloblasts, basophils, neutrophils, eosinophils, monocytes, granulocytes, dendritic cells, or macrophages.

11. A method of direct conversion of somatic cells into common myeloid progenitor (CMP) cells and macrophages, the method comprising preparing the CMP cells by culturing the somatic cells in media comprising the composition of claim 1.

12. The method of claim 11, wherein the media further comprise an HDAC inhibitor, a GSK-3 inhibitor, an antioxidant, or a combination thereof.

13. The method of claim 11, further comprising: conducting first culturing of the somatic cells in media comprising a TGF-β receptor inhibitor; and conducting second culturing of the cultured somatic cells in media comprising a TGF-β inhibitor and a GSK-3 inhibitor.

14. The method of claim 13, wherein one or more of the media of the first culturing or the media of the second culturing further comprise an HDAC inhibitor, an antioxidant, or a combination thereof.

15. The method of claim 11, wherein the somatic cells are one or more selected from fibroblasts, adipose stromal cells, epithelial cells, muscle cells, oral epithelial cells, somatic cells extracted from urine, blood cells, hair follicle stem cells, neural stem cells, hematopoietic stem cells, and mesenchymal stem cells.

16. The method of claim 10, wherein a duration of the culturing is 20 days to 36 days.

17. The method of claim 10, further comprising differentiating the prepared CMP cells into macrophages.

18. The method of claim 16, wherein in the differentiation into macrophages, the CMP cells are cultured in media comprising a macrophage colony stimulating factor (M-CSF), IL-4, or a combination thereof.

19. A composition comprising CMP cells or macrophages prepared by the method of claim 10.

20. The composition of claim 18, wherein the composition is formulated with a carrier for administration to a subject by topical application to the skin, oral administration, injection, in vivo transplantation, or a tissue-engineered matrix.
